(19) United States
(12) Patent Application Publication
(10) Pub. No.: US 2023/0333123 A1
(43) Pub. Date: Oct. 19, 2023

(54) LC-MS METHOD FOR DETECTING AND QUANTIFYING 11-OXYGENATED STEROIDS

(71) Applicant: Roche Diagnostics Operations, Inc., Indianapolis, IN (US)

(72) Inventors: Marius Fraefel, München (DE); Stefanie Grimm, München (DE); Thomas Hoffmann, Bad Heilbrunn (DE)

(21) Appl. No.: 18/337,018

(22) Filed: Jun. 18, 2023

Related U.S. Application Data
(63) Continuation of application No. PCT/EP2021/085808, filed on Dec. 15, 2021.

(30) Foreign Application Priority Data
Dec. 17, 2020 (EP) 20215188.2

(51) Int. Cl. G01N 33/74; G01N 30/72

(52) U.S. Cl. CPC G01N 33/743; G01N 30/7233

(57) ABSTRACT

The present invention relates to a method for detecting and/or quantifying one or more steroids using mass spectrometry, said steroids comprising at least one 11-oxygenated steroids. The method of the invention comprises (i) extracting the one or more steroids from the sample using solid phase extraction (SPE) so as to obtain an SPE extract comprising the one or more steroids; (ii) concentrating the one or more steroids, said concentrating comprising evaporating solvent from the SPE-extract; and (iii) detecting or quantifying the one or more steroids in the sample using mass spectrometry.

### FIELD OF THE INVENTION

The present invention relates to a method for detecting and/or quantifying one or more steroids using liquid chromatography coupled to mass spectrometry, said steroids comprising at least one 11-oxygenated C19 steroid.

### BACKGROUND OF THE INVENTION

Steroid levels are important diagnostic measures. For instance, an increase in androgenic steroids is a known defining feature of the polycystic ovary syndrome (PCOS), which is a collection of endocrine disorders affecting 4-10% of women in reproductive age.

Different methods, including immunoassay and mass spectrometry-based methods, have been used to detect levels of steroids that are of diagnostic value, such as androgenic steroids. While mass spectrometry-based methods have the potential to become the gold standard in steroid diagnostics, there is still a lack in harmonization of methods and a particular need to improve mass spectrometry based assays for the clinical routine laboratory (Stanczyk et al J Steroid Biochem Mol Biol. 2010. 121(3-5): p. 491-5). In particular, there is a need to provide mass spectrometry workflows for steroid diagnostics that are better suitable for a high throughput automatable analyses.

11-oxygenated C19 steroids represent a subgroup of C19 steroids that has recently attracted attention in the diagnosis of PCOS. A study by the group of O'Reilly et al (O'Reilly et al. J Clin Endocrinol Metab. 2017. 102(3): p. 840-848) found that besides classical androgen levels also certain 11-oxygenated C19 steroid levels are significantly increased in serum of PCOS patients and that the levels further correlate with markers of metabolic risk. The group of O'Reilly employed a liquid chromatography-tandem mass spectrometry (LC-MS/MS) method to measure 11-oxygenated C19 steroids in serum and urine samples. The employed method included the extraction of steroids including 11-oxygenated steroids from 400 μL of sample using 2 mL of methyl tert-butyl ether; i.e. by means of liquid extraction. Yet, liquid extraction involves large liquid volumes, considerable incubation times and a number of manual handling steps. Another limitation of the method of O'Reilly et al is that the measurement of certain steroids, such as dehydroepiandrosterone-sulfate (DHEAS), required a different and separately performed sample preparation workflow using ZnSO4

In another recent study, Yoshida and coworkers (Yoshida T et al., Endocrine Journal, 2018. 65(10): p. 979-990) measured four 11-oxygenated C19 steroids, namely 11-ketotestosterone (11KT), 11β-hydroxytestosterone (11OHT), 11β-hydroxyandrostenedione (11OHA4), and Ketoandrostenedione (11KA4), in blood samples of 28 PCOS patients and 31 eumenorrheic women using LC-MS/MS. The mass spectrometry workflow included extraction and derivatization steps. The detection method solely focused on 11-oxygenated C19 steroids. Levels of other steroids were not obtained in the same mass spectrometry workflow, but only from measurements of a separate study involving different mass spectrometry workflows and immunoassays.

Further studies measuring 11-oxygenated C19 steroids by LC-MS/MS based technologies have been described by Rege and coworkers (Rege et al., J Clin Endocrinol Metab, 2013, 98(3):1182-1188). Turcu and coworkers (Turcu et al., European Journal of Endocrinology, 2016, 174, 601-609), and Nanba and coworkers (Nanba et al., J Clin Endocrinol Metab, 2019, 104(7):2615-2622). These studies either use liquid extraction with the shortcomings mentioned above and/or involve different sample preparation methods for the measurement of different steroids.

Accordingly, there is a need to provide fast and reliable methods for detecting 11-oxygenated C19 steroids that are more suitable for mass spectrometry workflows involving fully automated sample preparation. In particular, there is also a need for the provision of mass spectrometry workflows for the measurement of 11-oxygenated C19 steroids with a sample preparation workflow that can also be used for the parallel detection of other steroids, such as, for example, androgens.

### SUMMARY OR THE INVENTION

The present invention relates to a method for detecting or quantifying one or more steroids in a sample using mass spectrometry. The method of the invention comprises:
a) extracting the one or more steroids from the sample using solid phase extraction (SPE) so as to obtain an SPE extract comprising the one or more steroids:
b) concentrating the one or more steroids, said concentrating comprising evaporating solvent from the SPE extract obtained in a); and
c) detecting or quantifying the one or more steroids in the sample using mass spectrometry.

The method of the invention allows detecting or quantifying one or more steroid analytes in a fast, automated and reliable manner.

As illustrated in the appended examples, the method of the invention can detect and quantify one or more 11-oxygenated C19 steroids and optionally other steroids (e.g. classical androgens) in a fast and sensitive manner. Thus, the present invention, in particular, provides a method for detecting and/or quantifying one or more 11-oxygenated C19 steroids and optionally one or more other steroids (e.g. classical androgens). A sample preparation workflow comprising the SPE of step a) and the concentration of step b) can enrich both 11-oxygenated C19 steroids and other steroids (e.g. classical androgens) and allo %% s for a robust measurement of those in a single workflow involving a common sample preparation.

In embodiments, the SPE of step a) uses microparticles (in particular magnetic microparticles) as solid phase. These microparticles are in particular capable of adsorbing and/or binding the one or more steroids present in the sample.

The SPE may in particular be a batch-type SPE, involving binding the one or more steroids to the microbeads, optionally washing the microbeads and eluting the one or more steroids from the microbeads to obtain an SPE extract obtaining the one or more steroids to be detected or quantified.

Accordingly, in specific embodiments the present invention relates to a method for detecting or quantifying one or more steroids in a sample, said method comprising:
a) extracting the one or more steroids from the sample using a magnetic particle based batch-type SPE so as to obtain an SPE extract comprising the one or more steroids;
b) concentrating the one or more steroids, said concentrating comprising evaporating solvent from the SPE-extract obtained in a); and
c) detecting or quantifying the one or more steroids in the sample using mass spectrometry.

The present invention in particular also relates to the following items:
1. A method for detecting and/or quantifying one or more steroids in a sample using mass spectrometry, said method comprising:
   a) extracting the one or more steroids from the sample using solid phase extraction (SPE) so as to obtain an SPE extract comprising the one or more steroids;
   b) concentrating the one or more steroids, said concentrating comprising evaporating solvent from the SPE-extract obtained in a); and
   c) detecting and/or quantifying the one or more steroids in the sample using mass spectrometry.
   wherein the one or more steroids comprise one or more 11-oxygenated C19 steroids.
2. The method of item 1, wherein the one or more 11-oxygenated C19 steroids are selected from the group consisting of 11β-Hydroxyandrostenedione (11-OHA4), 11-Ketotestosterone (11KT), 11-Ketoandrostenedione (11KA4), and 11β-Hydroxytestosterone (11OHT)
3. The method of item 1 or 2, wherein said one or more steroids comprise one or more steroids selected from the group consisting of Testosterone (T), Androstenedione (A4), Dehydroepiandrosterone (DHEA), Dehydroepiandrosterone sulfate (DHEAS) and Epitestosterone (ET).
4. The method of any one of items 1 to 3, wherein the one or more steroids comprise 11β-Hydroxyandrostenedione (11-OHA4), 11-Ketotestosterone (11KT), 11-Ketoandrostenedione (11 KA4), 11β-Hydroxytestosterone (11OHT), Testosterone (T), Androstenedione (A4), Dehydroepiandrosterone (DHEA), Dehydroepiandrosterone sulfate (DHEAS) and Epitestosterone (ET).
5. The method of any one of items 1 to 4, wherein the sample is a fluid, in particular a biological fluid.
6. The method of any one of items 1 to 5, wherein the sample is an obtained body fluid (e.g. human body fluid) selected from the group consisting of serum and plasma.
7. The method of any one of items 1 to 6, wherein the sample is an obtained serum sample.
8. The method of any of items 1 to 7, wherein the sample volume subjected to the SPE in a) is less than 500 μl, in particular less than 250 μl, in particular less than 200 μl, in particular 150 μl.
9. The method of any one of items 1 to 8, wherein said method further comprises a pretreatment step prior to step a) comprising adding acetonitrile (ACN) and/or methanol (MeOH) to the sample.
10. The method of any one of items 1 to 9, wherein pretreatment step comprises adding ACN to the sample at a final concentration of 1 to 10 vol %, in particular 2 to 5 vol %, in particular 2 vol % ACN.
11. The method of item 9 or 10, wherein the one or more steroids comprise Dehydroepiandrosterone sulfate (DHEAS) and/or Testosterone (T).
12. The method of any one of items 1 to 11, w herein the SPE comprises.
   a) capture the one or more steroids to a solid phase;
   b) optionally one or more wash steps of the solid phase; and
   c) eluting the one or more steroids from the solid phase.
13. The method of any one of items 1 to 12, wherein the SPE is a batch-type SPE.
14. The method of any one of items 1 to 13, wherein the solid phase used for the SPE is formed by magnetic particles, in particular magnetic microbeads.
15. The method of any one of items 1 to 14, wherein the solid phase of the SPE is formed by particles (e.g. magnetic particles), said particles being configured to capture the one or more steroids from the sample and to release said one or more steroids when treated with an elution solvent.
16. The method of any one of items 1 to 15, wherein the solid phase of the SPE is formed by particles each comprising a porous polymer matrix, in particular magnetic particles comprising a magnetic core and a porous polymer matrix.
17. The method of item 16, wherein the porous polymer matrix comprises pores having a pore size smaller than 100 nm, in particular smaller than 90 nm, in particular smaller than 80 nm, in particular smaller than 70 nm, in particular smaller than 60 nm, in particular in the range from 0.5 nm to 50 nm as determined according to ISO15901.
18. The method of item 16 or 17, wherein the porous polymer matrix comprises a crosslinked polymer, wherein the polymer preferably comprises a co-polymer obtained or obtainable by a method comprising a polymerization of at least two different monomeric building blocks selected from the group consisting of styrene, functionalized styrenes, vinylbenzylchloride, divinylbenzene, vinylacetate, methylmethaacrylate and acrylic acid.
19. The method of any one of items 16 to 18, wherein the particle is a hypercrosslinked particle, wherein the porous polymer is hypercrosslinked.
20. The method of item 19, wherein the hypercrosslinking of the porous polymer is a hypercrosslinking obtainable by a Friedel-Crafts reaction.
21. The method of any one of items 16 to 20, wherein the surface of the porous polymer matrix is functionalized with at least an —OH, in particular an —OH.
22. The method of any one of items 16 to 20, wherein the porous polymer matrix comprises nitrogen atoms, in particular positively charged nitrogen atoms.
23. The method ofany one of items 19, 20, and 22, wherein the hypercrosslinking is achieved with a hypercrosslinking bond that comprises at least two nitrogen atoms, in particular wherein the hypercrosslinking bond comprises a diamine.
24. The method of item 23, wherein the hypercrosslinking bond comprises at least one (e.g. two) positively charged nitrogen atom.
25. The method of any one of items 19, 20 and 22 to 24, wherein the hypercrosslinking is achieved with a hypercrosslinking bond that consists of a molecule comprising at least two nitrogen atoms within its structure which are part of the hypercrosslinking bond, wherein the molecule comprising at least two nitrogen atoms within its structure has the general structure of formula 1

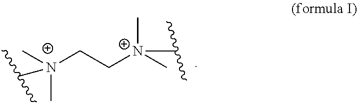 [SMILES: *[N+](C)(C)CC[N+](*)(C)C]

26 The method of any one of items 12 to 25, wherein the capturing of the one or more steroids to the solid phase comprises incubating the solid phase with the sample for 4 to 15 min, in particular 9 min under conditions that allow capturing the one or more steroids.
27. The method of item 26, wherein said incubating of the solid phase with the sample is performed at a temperature of 25 to 45° C. in particular 35 to 40° C. in particular 37° C.
28. The method of any of items 12 to 27, wherein the SPE comprises one or more (e.g. two) wash steps using a wash solution.
29. The method of item 28, wherein the wash solution has a pH of 2 to 4, in particular of 2.6.
30. The method of any one of items 26 to 29, wherein the SPE is a batch-type SPE and wherein the one or more wash steps comprise
   adding the wash buffer to the solid phase;
   mixing the solid phase (e.g. microparticles, in particular magnetic microbeads) with the wash buffer and incubating the mixture in particular for 3 to 10 sec, in particular 4 sec;
   pelleting the solid phase (e.g. via magnetic force if magnetic microbeads are used as solid phase); and
   removing the supernatant.
31. The method of any one of items 12 to 30, wherein eluting the one or more steroids comprises adding an elution solvent to the solid phase.
32. The method of any one of items 12 to 31, wherein eluting the one or more steroids comprises incubating the solid phase in presence of the added elution solvent.
33. The method of item 32, wherein the incubation of the solid phase in presence of the elution solvent is conducted for 30 to 400 sec, in particular 50 to 150 sec, in particular 108 sec.
34. The method of any one of items 31 to 33, wherein the volume of the added elution solvent is 50 to 150% of the initial sample volume, in particular 100 to 120%.
35. The method of any one of items 31 to 34, wherein the elution solvent comprises ACN, in particular at a concentration of 40 to 100 vol %, in particular 45 to 90 vol %, in particular 60 to 80 vol %.
36. The method ofany one of items 31 to 35, wherein the elution solvent comprises MeOH, in particular at a concentration of 70 to 100 vol %, in particular 80 to 90 vol %, in particular 80 vol %.
37. The method of any one of items 1 to 36, wherein said method further comprises adding an internal standard (ISTD) to the sample, preferably prior to the extraction using SPE and an optional pretreatment.
38. The method of item 37, wherein the ISTD is a heavy isotope labeled form of the steroid to be quantified.
39. The method of any one of items 1 to 38, wherein said evaporating of the solvent of the SPE extract results in a liquid volume reduction of 50 to 100%, in particular 60 to 85%.
40. The method of item 39, wherein the concentrating further comprises adjusting the volume after evaporation by adding a diluent solution to 10 to 40%, in particular 20 to 40%, in particular 24 to 28% of the volume of the sample subjected to SPE (e.g. as defined in item 8).
41. The method of item 40, wherein the diluent solution comprises water or 10-30 vol % of methanol, in particular 20 vol % of methanol or acetonitril
42. The method of item 40 or 41, wherein the organic solvent concentration (e g of acetonitrile or methanol) in the SPE extract is reduced by the concentrating such that is compatible with liquid chromatography (LC) used in the LC-MS analysis, in particular to a concentration of methanol or acetonitrile equal or lower than 30 vol %, in particular to a concentration of methanol or acetonitrile equal or lower than 20 vol %.
43. The method of any one of items 1 to 42, wherein the mass spectrometry is coupled to liquid chromatography (e.g. wherein the analysis is a LC-MS analysis or more specifically a LC-MS/MS analysis).
44. The method of item 43, wherein the liquid chromatography is HPLC.
45. The method of item 44, wherein the HPLC separation principle is a reversed phase HPLC (RP-HPLC) or rapid LC.
46. The method of item 45, wherein the RP-HPLC comprises a C18-based stationary phase for separation.
47. The method of item 45 or 46, wherein the RP-HPLC comprises a gradient elution
48. The method of item 47, wherein the elution gradient is linear and is established within 1-2 min, in particular 1.1-1.5 min, in particular 1.2 min.
49. The method of any one of items 1 to 48, wherein the ion formation of the mass spectrometer is based on electrospray ionization (ESI) in positive mode.
50. The method of any of items 1 to 49, wherein the MS device used for the mass spectrometry is a tandem mass spectrometer, in particular a triple quadrupole device.
51. The method of any one of items 1 to 50, wherein the method is automated.
52. The method of any one of items 1 to 51, wherein the method is performed in a random-access compatible system.
53. The method of any one of items 1 to 52, wherein the mass spectrometry comprises Multiple Reaction Monitoring Mass Spectrometry (MRM-MS).
54. The method of any one of items 1 to 53, wherein for each of the one or more steroids and/or ISTD(s) two fragment ions are detected by mass spectrometry, wherein one of the two fragment ions is used for quantification and the second fragment ion is used as a qualifier.
55. The method of any one of items 1 to 54, wherein the one or more steroids comprise 11OHA4, and wherein for 11OHA4 a positively charged parent ion having an m/z value of 303.1±0.5 is generated and selected for fragmentation.
56. The method of item 55, wherein a first positively charged 11OHA4-fragment ion having an m/z ratio of 267.2±0.5 and/or a second positively charged 11OHA4-fragment ion having an m/z ratio of 121.1±0.5 are generated by fragmentation of the parent ion selected for 11OHA4 and wherein said first and/or second 11OHA4-fragment ion(s) is/are detected by the mass spectrometry and is/are optionally used for quantification.
57. The method of item 56, wherein the first 11OHA4-fragment ion with an m/z ration of 267.2±0.5 is generated, detected and used for quantification of 11OHA4.
58. The method of any one of items 1 to 57, wherein the one or more steroids comprise 11KT, and wherein for 11KT a positively charged parent ion having an m/z value of 303.1±0.5 is generated and selected for fragmentation.
59. The method of item 58, wherein a first positively charged 11KT-fragment ion having an m/z ratio of 121.1±0.5 and/or a second positively charged 11KT-fragment ion having an m/z ratio of 259.2±0.5 are generated by fragmentation of the parent ion selected for 11KT, and wherein said first and/or second 11KT-fragment ion is/are detected by the mass spectrometry and is/are optionally used for quantification.
60. The method of item 59, wherein the first 11KT-fragment ion with an m/z ration of 121.1±0.5 is generated, detected and used for quantification of 11KT.
61. The method of any one of items 1 to 60, wherein the one or more steroids comprise 11KA, and wherein for 11KA a positively charged parent ion having an m/z value of 301.05±0.50 is generated and selected for fragmentation.
62. The method of item 61, wherein a first positively charged 11KA-fragment ion having an m/z ratio of 257.2±0.5 and/or a second positively charged 11KA-fragment ion having an m/z ratio of 121.2±0.5 are generated by fragmentation of the parent ion selected for 11KA, and wherein said first and/or second 11KA-fragment ion is/are detected in the by the mass spectrometry and is/are optionally used for quantification.
63. The method of item 62, wherein the first 11KA-fragment ion with an m/z ration of 257.2±0.5 is generated, detected and used for quantification of 11KA.
64. The method of any of items 1 to 63, wherein the one or more steroids comprise 11OHT, and wherein for 11OHT a positively charged parent ion having an m/z value of 305.2±0.5 is generated and selected for fragmentation
65. The method of item 64, wherein a first positively charged 11OHT-fragment ion having an m/z ratio of 269.2±0.5 and/or a second positively charged 11OHT-fragment ion having an m/z ratio of 121.1±0.5 are generated by fragmentation of the parent ion selected for 11OHT, and wherein said first and/or second 11OHT-fragment ion is/are detected in the by the mass spectrometry and is/are optionally used for quantification.
66. The method of item 65, wherein the first 11OHT-fragment ion with an m/z ration of 269.2±0.5 is generated, detected and used for quantification of 11OHT.
67. The method of any one of items 1 to 66, wherein the one or more steroids comprise T, and wherein for T a positively charged parent ion having an m/z value of 289.2±0.5 is generated and selected for fragmentation.
68. The method of item 67, wherein a first positively charged T-fragment ion having an m/z ratio of 97.1±0.5 and/or a second positively charged T-fragment ion having an m/z ratio of 109.1±0.5 are generated by fragmentation of the parent ion selected for T, and wherein said first and/or second T-fragment ion is/are detected by the mass spectrometry and is/are optionally used for quantification.
69. The method of item 68, wherein the first T-fragment ion with an m/z ration of 97.1±0.5 is generated, detected and used for quantification of T
70. The method of any one of items 1 to 69, wherein the one or more steroids comprise A4, and wherein for A4 a positively charged parent ion having an m/z value of 287.2±0.5 is generated and selected for fragmentation.
71. The method of item 70, wherein a first positively charged A4-fragment ion having an m/z ratio of 97.1±0.5 and/or a second positively charged A4-fragment ion having an m/z ratio of 109.1±0.5 are generated by fragmentation of the parent ion selected for A4, and wherein said first and/or second A4-fragment ion is/are detected by the mass spectrometry and is/are optionally used for quantification.
72. The method of item 71, wherein the first A4-fragment ion with an m/z ration of 97.1±0.5 is generated, detected and used for quantification of A4.
73. The method of any one of items 1 to 72, wherein the one or more steroids comprise DHEA, and wherein for DHEA a positively charged parent ion having an m/z value of 289.2±0.5 is generated and selected for fragmentation.
74. The method of item 73, wherein a first positively charged DHEA-fragment ion having an m/z ratio of 213.1±0.5 and/or a second positively charged DHEA-fragment ion having an m/z ratio of 91.0±0.5 are generated by fragmentation of the parent ion selected for DHEA, and wherein said first and/or second DHEA-fragment ion is/are detected by the mass spectrometry and is/are optionally used for quantification.
75. The method of item 74, wherein the first DHEA-fragment ion with an m/z ration of 213.1±0.5 is generated, detected and used for quantification of DHEA.
76. The method of any one of items 1 to 75, wherein the one or more steroids comprise DHEAS, and wherein for DHEAS a negatively charged parent ion having an m/z value of 367.2±0.5 is generated and selected for fragmentation.
77. The method of item 76, wherein a first negatively charged DHEAS-fragment ion having an m/z ratio of 80.1±0.5 and/or a second negatively charged DHEAS-fragment ion having an m/z ratio of 97.1±0.5 are generated by fragmentation of the parent ion selected for DHEAS, and wherein said first and/or second DHEAS-fragment ion is/are detected by the mass spectrometry and is/are optionally used for quantification.
78. The method of item 77, wherein the first DHEAS-fragment ion with an m/z ration of 80.1±0.5 is generated, detected and used for quantification of DHEAS.
79. The method of any one of items 1 to 78, wherein the one or more steroids comprise ET, and wherein for ET a positively charged parent ion having an m/z value of 289.2±0.5 is generated and selected for fragmentation.
80. The method of item 79, wherein a first positively charged ET-fragment ion having an m/z ratio of 109.0±0.5 and/or a second positively charged ET-fragment ion having an m/z ratio of 97.0±0.5 are generated by fragmentation of the parent ion selected for ET, and wherein said first and/or second ET-fragment ion is/are detected by the mass spectrometry and is/are optionally used for quantification.
81. The method of item 80, wherein the first ET-fragment ion with an m/z ration of 109.0±0.5 is generated, detected and used for quantification of ET.

### DETAILED DESCRIPTION OF THE INVENTION

The present invention relates to a method for detecting or quantifying one or more steroids in a sample using mass spectrometry. The method comprises:
a) extracting the one or more steroids from the sample using solid phase extraction (SPE) so as to obtain an SPE extract comprising the one or more steroids;
b) concentrating the one or more steroids, said concentrating comprising evaporating solvent from the SPE-extract obtained in a); and
c) detecting and/or quantifying the one or more steroids in the sample using mass spectrometry (e.g. LC-MS/MS).

As demonstrated by the appended examples the method of the invention allows for sensitive, fast and reliable detection of 11-oxygenated C19 steroids and other steroids. Thus, in particular embodiments, the one or more steroids may comprise one or more (e.g. 1, 2, 3 or 4) 11-oxygenated C19 steroids. In one embodiment, the one or more steroids to be detected and/or quantified may consist of one or more 11-oxygenated C19 steroids (e.g. 1, 2, 3 or 4). In another embodiment, the one or more steroids may comprise one or more 11-oxygenated C19 steroids (e.g. 1, 2, 3 or 4) and one or more other steroids (i.e. one or more steroids that are not 11-oxygenated C19 steroids). In embodiments, the method of the invention may comprise detecting and/or quantifying at least two, at least three or at least four 11-oxygenated C19 steroids. 11-oxygenated C19 steroids are known in the an and the method of the invention may involve detection of one or more of the 11-oxygenated C19 steroids known in the art. Exemplary but non-limiting 11-oxygenated C19 steroids known in the an are 11β-Hydroxyandrostenedione (11-OHA4), 11-Ketotestosterone (11KT), 11-Ketoandrostenedione (11KA4), and 11β-Hydroxytestosterone (11OHT), 11-ketodihydrotestosterone (11 KDHT) and 11β-hydroxydihydrotestosterone (11OHDHT). Accordingly, the method of the invention may comprise detecting and/or quantifying one or more 11-oxygenated C19 steroids. In particular, the one or more 11-oxygenated C19 steroids to be detected and/or quantified in the context of the invention may be selected from the group consisting of 11β-Hydroxyandrostenedione (11-OHA4), 11-Ketotestosterone (11KT), 11-Ketoandrostenedione (11KA4), and 11β-Hydroxytestosterone (11OHT). In some embodiments, the one or more steroids to be detected and/or quantified by the method of the invention may consist of 11β-Hydroxyandrostenedione (11-OHA4), 11-Ketotestosterone (11KT), 11-Ketoandrostenedione (11KA4), and 11β-Hydroxytestosterone (11OHT).

As demonstrated by the appended Examples, the method of the invention can detect and quantify a panel of steroids using a mass spectrometry workflow comprising a unitary sample preparation workflow. Detection of several steroids with a unitary mass spectrometry workflow has the advantage to reduce analysis time, reduces handling steps, reduces the required sample volume, increases throughput and facilitates automation. These aspects facilitate diagnosis of medical conditions related to alterations in steroid levels.

The one or more steroids to be detected or quantified in the context of the method of the invention may in particular comprise steroids selected from the group consisting of cortisol, dehydroepiandrosterone (DHEA), dehydroepiandrosterone sulfate (DHEAS), estradiol, progesterone, testosterone, 17-hydroxyprogesteron, aldosterone, Androstenedione (A4), dihydrotestosterone and Epitestosterone (ET) and 11-oxygenated C19 steroids. In particular embodiments, the one or more steroids may be selected from the group consisting of Testosterone (T), Androstenedione (A4), Dehydroepiandrosterone (DHEA), Dehydroepiandrosterone sulfate (DHEAS), Epitestosterone (ET) and 11-oxygenated C19 steroids. In particular embodiments the one or more steroids may be selected from the group consisting of Testosterone (T), Androstenedione (A4), Dehydroepiandrosterone (DHEA), Epitestosterone (ET) and 11-oxygenated C19 steroids

In embodiments, the one or more steroids to be quantified and/or detected may comprise (i) one or more 11-oxygenated C19 steroids selected from the group consisting of 11β-Hydroxyandrostenedione (11-OHA4), 11-Ketotestosterone (11KT), 11-Ketoandrostenedione (11 KA4) and 11β-Hydroxytestosterone (11 OHT) and (ii) one or more other steroids selected from the group consisting of cortisol, dehydroepiandrosterone (DHEA), dehydroepiandrosterone sulfate (DHEAS), estradiol, progesterone, testosterone, 17-hydroxyprogesteron, aldosterone, Androstenedione (A4), dihydrotestosterone and Epitestosterone (ET). In particular embodiments, the one or more steroids to be quantified and/or detected may comprise (i) one or more 11-oxygenated (C19 steroids selected from the group consisting of 11β-Hydroxyandrostenedione (11-OHA4), 11-Ketotestosterone (11 KT), 11-Ketoandrostenedione (11KA4) and 11β-Hydroxytestosterone (11OHT) and (ii) one or more other steroids selected from the group consisting of Testosterone (T). Androstenedione (A4), Dehydroepiandrosterone (DHEA), Dehydroepiandrosterone sulfate (DHEAS) and Epitestosterone (ET). In particular embodiments, the one or more steroids to be quantified and/or detected may comprise (i) one or more 11-oxygenated C19 steroids selected from the group consisting of 11β-Hydroxyandrostenedione (11-OHA4), 11-Ketotestosterone (11KT), 11-Ketoandrostenedione (11 KA4) and 11β-Hydroxytestosterone (11OHT) and (ii) one or more other steroids selected from the group consisting of Testosterone (T), Androstenedione (A4). Dehydroepiandrosterone (DHEA) and Epitestosterone (ET).

In embodiments, the one or more steroids to be quantified and/or detected may consist of (i) one or more 11-oxygenated (C19 steroids selected from the group consisting of 11β-Hydroxyandrostenedione (11-OHA4), 11-Ketotestosterone (11KT), 11-Ketoandrostenedione (11 KA4) and 11β-Hydroxytestosterone (11OHT) and (ii) one or more steroids of the group consisting of Testosterone (T), Androstenedione (A4). Dehydroepiandrosterone (DHEA), Dehydroepiandrosterone sulfate (DHEAS) and Epitestosterone (ET). In embodiments, the one or more steroids to be quantified and/or detected may consist of (i) one or more 11-oxygenated C19 steroids selected from the group consisting of 11β-Hydroxyandrostenedione (11-OHA4), 11-Ketotestosterone (11KT), 11-Ketoandrostenedione (11KA4) and 11β-Hydroxytestosterone (11OHT) and (ii) one or more steroids of the group consisting of Testosterone (T). Androstenedione (A4), Dehydroepiandrosterone (DHEA) and Epitestosterone (ET).

In particular embodiments, the one or more steroids may comprise 11β-Hydroxyandrostenedione (11-OHA4), 11-Ketotestosterone (11KT), 11-Ketoandrostenedione (11KA4), 11β-Hydroxytestosterone (11OHT), Testosterone (T). Androstenedione (A4). Dehydroepiandrosterone (DHEA), Dehydroepiandrosterone sulfate (DHEAS) and Epitestosterone (ET). The appended Examples demonstrate that the method of the invention allows for efficient detection and quantification of all these steroids in a single mass spectrometry run.

In principle, any sample that comprises or is suspected to comprise steroids may be subjected to the method of the invention. While a sample subjected to solid phase extraction in step a) needs to be liquid, in principle also solid samples (e.g. dried blood spots) can be subjected to the method of the invention. For solid samples, an additional sample preparation step prior to SPE is included, which comprises reconstituting the dried blood spot in a liquid. Respective methods and means for reconstituting a solid sample into a liquid such that steroids are recovered in the liquid are known in the art (Rossi et al, Clin Chem Lab Med 2011:49(4):677-684; Kim et al., Ann Lab Med 2015; 35.578-585).

The method of the invention uses a previously obtained sample. Thus, the method of the invention is an in vitro method. The sample may in particular be a sample derived from a human individual. In a particular embodiment, the sample may be derived from a female. The female may suffer and/or may be suspected to suffer from PCOS.

In embodiments, the sample may be a liquid sample, e.g. a biological fluid. In particular embodiments, the sample may be a body fluid. Exemplary but non-limiting examples for body fluids are whole blood, serum, plasma, urine, seminal fluid, (female) follicular fluid and salvia. In particular embodiments, the sample may be a blood sample selected from whole blood, serum and plasma. In specific embodiments, the sample may be selected from the group consisting of serum, plamsa and urine. In specific embodiments, the sample may be urine. In a preferred embodiment, the sample is serum or plasma. Depending on the sample type used a sample preparation step may be used and the amount of sample may be adjusted. A skilled person in the art is aware how to conduct such sample preparation.

The sample volume subjected to the SPE in step a) of the method can be varied depending on the sample type and the type and/or concentration of the steroids to be detected in the sample. It is an advantage of the method of the invention that it can determine the presence and/or quantity of the one or more steroids using a low sample volume of, e.g. 100 μl to 500 μl, or more particularly 100 μl to 350 μl. Low sample volumes have the advantage to reduce the reagent volumes for the analysis and the total analysis time (e.g. by reducing the time for liquid chromatography etc.) and offer the opportunity to subject extra sample material to different analyses. The combination of SPE extraction and the concentration step of the method of the present invention contribute to an increased sensitivity and, thus, that sample volumes can be kept low.

For instance, the sample volume may be less than 500 μl, in particular less than 350 μl in particular less than 250 μl, in particular less than 200 μl, in particular 150 μl. In embodiments, a sample volume of 150 μl to 500 μl or more particularly 150 μl to 200 μl may be used.

Depending on the sample type, a fraction of the one or more steroids or one or more of the steroids to be detected and/or quantified may form a complex with one or more proteins (e.g. a steroid binding protein and/or sex hormone-binding globin) or other sample constituents (e.g. serum constituents or albumin). The method of the invention may comprise a step of releasing the one or more steroids to be detected and/or quantified from binding partners such as proteins (e g sex hormone-binding globin). The releasing step may in particular be conducted prior to SPE. Performing a releasing/pretreatment step can enhance the availability of one or more steroids for the detection and/or quantification. The pretreatment step may be a “deproteinization” step. i.e. a step that releases the some or all of the one or more steroids from one or more proteins to which they are bound.

The pretreatment/releasing step may comprise adding a releasing agent (e.g. deproteinization agent) to the sample. A releasing agent (e.g. deproteinization agent) is an agent that releases the one or more steroids from its binding partner(s) in the sample (proteins such as sex hormone-binding globin and/or other constituents of the sample) when added to a sample. In some embodiments, a releasing composition (e.g. deproteinization composition) may be used. A releasing composition (e.g. deproteinization composition) is a mixture of two or more substances comprising at least one agent that triggers release of the one or more steroids from its binding partner(s)(proteins such as sex hormone-binding globin and/or other constituents of the sample) when added to the sample. Deproteinization agents and compositions as well as techniques using the same are known in the art. In the context of the present disclosure a releasing agent (e.g. deproteinization agent) may be an organic solvent such as, for example, an organic solvent selected from acetonitrile (ACN), methanol (MeOH), and dimethylsulfoxid (DMSO). Exemplary but non-limiting releasing compositions (e.g. deproteinization compositions) include but are not limited to mixtures comprising at least two from the group of acetonitrile (ACN), methanol (MeOH), and dimethylsulfoxid (DMSO). The volume of the releasing agent (e.g. deproteinization agent) and/or releasing composition (e.g. deproteinization composition) added to the sample may be adjusted depending on the agent and/or agent composition and the type of binding that it should interfere with For instance, ACN may be added to the sample such that a final concentration of 1 to 10 vol %, in particular 2 to 5 vol % and in particular about 2 vol % or exactly 2 vol %. MeOH may, for example, be added to the sample to the sample at a final concentration of 2.5-30 vol %, in particular 5-15 vol % and in particular 7.5 vol %. DMSO may be added to the sample at a final concentration of 2-20 vol %, in particular 3-10 vol % and in particular 5 vol %. The pretreatment step in the context of the invention may further or alternatively involve lowering or increasing the pH of the sample. It is known in the art that a change in pH can interfere with binding of steroids to sample constituents. For instance, the pretreatment step may comprise adjusting the sample pH to apH of 2 to 5, 3 to 4 or in particular 2.6. For acidification, an acid such as formic acid (FA) may be used.

In preferred embodiments, the method of the invention may comprise a pretreatment step prior to step a) comprising adding an organic solvent as releasing agent (e.g., at neutral pH). For instance, the organic solvent acetonitrile (ACN) may be added to the sample at a final concentration as indicated above. As demonstrated in the appended Examples, using an organic solvent such as ACN facilitates detection of one or more steroids and can increase the measured concentration. For instance, the appended Examples demonstrate that pretreatment with an organic solvent such as ACN facilitates detection and/or quantification of at least dehydroepiandrosterone sulfate (DHEAS) and/or testosterone (T), respectively. Accordingly, in embodiments of the present disclosure, the one or more steroids to be detected and/or quantified may comprise DHEAS and/or testosterone (and optionally one or more 11-oxygenated C19 steroids), and the method may comprise a pretreatment (in particular a deproteinization) step. The pretreatment step may comprise adding an organic solvent to the sample prior to solid phase extraction.

The pretreatment typically further involves incubating the sample for a defined time before proceeding with solid phase extraction. The duration of the incubation may be 10 to 900 sec. in particular 30 to 900 sec and in particular 50 to 900 sec.

In embodiments, also pretreatment conditions as known in the art may be employed. Non-limiting examples for pretreatment conditions are described in Gervasoni, J., et al. (Clin Biochem, 2016., 49(13-14): p. 998-1003), which is herein incorporated by reference in its entirety.

The method of the invention may comprise the step of “extracting the one or more steroids from the sample using solid phase extraction (SPE) so as to obtain an SPE extract comprising the one or more steroids”. “Extracting the one or more steroids from the sample” means that the sample complexity is reduced; i.e. that the one or more steroids are separated/purified partially or fully from other sample constituents. In the context of the invention, the one or more steroids are in particular extracted in the same SPE workflow. The reduction of sample complexity by the extraction facilitates mass spectrometry analyzes of the one or more steroids and reduces background signal. In embodiments, the step of “extracting the one or more steroids from the sample” may be “enriching the one or more steroids from the sample”. “Enriching” in this context means that a SPE extract is generated in which the amount(s) of the one or more steroids is/are increased relative to other sample constituents. In embodiments, the relative abundance relative to at least one other sample constituent. e.g. a sample constituent that may negatively affect the signal to noise ratio, may be increased.

“Solid phase extraction” (SPE) as used in the context of the present invention refers to a method that partially or fully separates an analyte or a group of analytes from other compounds comprised in a liquid mixture and/or sample, said method relying on a differential solid phase and liquid phase distribution of the analyte(s) and one or more of the other compounds comprised in a mixture and/or sample. In a first embodiment of solid phase extraction according to the present disclosure, the analyte(s) may have higher binding affinity to a solid phase than one or more of the other compounds in a mixture or sample. By the higher binding affinity of analyte(s) to the solid phase the analyte(s) may be partially or fully separated, i.e. extracted, from the other compounds of the mixture and/or sample. In a second alternative embodiment, the analyte may have a binding affinity to a solid phase that is lower than the binding affinity of one or more other compounds comprised in the mixture and/or sample subjected to solid SPE. In this embodiment, the one or more steroids remain in the liquid phase and one or more other sample constituents are removed by binding to the solid phase. Accordingly, the term solid phase extraction, as used herein, includes different embodiments, such as: (i) retention of the analyte(s) on a solid phase that allows partial or full removal of other compounds with the liquid phase (optionally also with one or more wash steps) and (ii) retention of other compounds on the solid phase and extraction of the analyte in the liquid phase. In the embodiments (i). SPE typically involves an elution using a suitable elution solution to release the reversibly bound analyte(s) from the solid phase. The elution solution can be selected dependent on the binding principle of the solid phase.

The “solid phase extraction” as used in the context of the present invention includes but is not limited to techniques such as classical solid phase extraction methods using a solid phase extraction cartridge/column or a solid phase tip. In particular, the term “solid phase extraction” in the context of the invention includes particle-based in particular bead based workflows. The term “solid phase extraction” in the context of the present disclosure includes different separation principles. In embodiments, the analytes (i.e. the one or more steroids) may be retarded by the solid phase (e.g. beads) and one or more other sample constituents remain in the liquid phase. In alternative embodiments, the analytes (i.e. the one or more steroids) may remain in the liquid phase and one or more other sample constituents bind to the solid phase.

The “solid phase” used for solid phase extraction in the context of the invention includes but is not limited to a surface or particles (e.g. microparticles such as microbeads). In a particular embodiment, the solid phase may be beads, in particular microbeads. Beads (e.g. microbeads) may be non-magnetic, magnetic, or paramagnetic. In a particularly preferred embodiment, the solid phase may be magnetic microbeads. The beads (e.g. microbeads, in particular magnetic microbeads) may be made of various different materials. The beads (e.g. magnetic beads) may have various sizes (e.g. in the μm range) and comprise a surface with or without pores.

In a particular embodiment of the invention, the solid phase may be a dispensable solid phase; i.e. a solid phase material that can be held in suspension and dispensed. Non-limiting examples for such dispensable solid phases are particles, in particular beads, more particularly microbeads and even more particularly magnetic microbeads. A dispensable solid phase has the advantage that it can be efficiently used in a random access mode on an automated sample preparation and mass spectrometry analyser, which may require using different solid phases for different analytes. Moreover, the amount of a dispensable solid phase material can be more easily adjusted.

The solid phase (e.g. beads, in particular microbeads, even more particularly magnetic microbeads) may be coated in a manner that allows binding/capturing of the one or more steroids. Suitable coatings for capturing/binding the one or more steroids can be selected based on prior art knowledge.

In embodiments, the solid phase (e.g. beads, in particular microbeads, even more particularly magnetic microbeads) may comprise antibodies or fragments thereof specifically binding to said one or more steroids attached to the surface. In a particular embodiment immunobeads (i.e. magnetic particles such as microbeads having antibodies or antigen-binding fragments thereof attached to the surface) binding/capturing the one or more steroids may be employed for the SPE. In other embodiments, the solid phase may be coated with a porous polymer matrix that allows retardation of the one or more steroids.

In embodiments, the solid phase used for the SPE may be coated with a nitrogen-comprising polymer having a permanent positive charge. In a particular embodiment, the solid phase may be microbeads that are coated on their surface with a nitrogen comprising polymer having a permanent positive charge. In embodiments, these microbeads may have a magnetic core; i.e. may be magnetic beads.

In embodiments, the solid phase may be magnetic microbeads comprising a magnetic core and a polystyrene layer around the magnetic core. The polystyrene layer may have chemical modifications (e.g. nitrogen comprising substituents with a permanent charge).

In embodiments, the solid phase of the SPE may be formed by particles (e.g. magnetic particles), in particular the solid phase may consist of particles (e.g. magnetic particles). These particles may be configured to capture the one or more steroids from the sample and to release said one or more steroids when treated with an elution solvent. At least some other sample constituents are either less efficiently, or not captured by those magnetic beads, such that the SPE separates the one or more steroids to a certain degree from other sample constituents.

In embodiments, particles as described in WO2018189286A1 or WO2019141779A 1 may be used for the SPE to capture the one or more steroids from the sample. These documents and particularly the particles or beads as described therein are incorporated herein in their entirety.

In embodiments, particles may comprise a porous polymer matrix. Optionally the particles may be magnetic and may further comprise a magnetic core. The polymer matrix typically embeds the magnetic core.

The porous polymer matrix may comprise pores having a pore size smaller than 100 nm, in particular smaller than 90 nm, in particular smaller than 80 nm, in particular smaller than 70 nm, in particular smaller than 60 nm, in particular in the range from 0.5 nm to 50 nm as determined according to ISO15901.

The porous polymer matrix may comprise a crosslinked polymer. The polymer may preferably comprises a co-polymer obtained or obtainable by a method comprising a polymerization of at least two different monomeric building blocks selected from the group consisting of styrene, functionalized styrenes, vinylbenzylchloride, divinylbenzene, vinylacetate, methylmethaacrylate and acrylic acid. In particular, the polymer matrix may comprise a co-polymer obtained or obtainable by a method comprising a polymerization of vinylbenzylchloride and divinylbenzene.

In embodiments, the surface of the porous polymer matrix may be functionalized, i.e. modified with a functional group.

In embodiments, the surface of the porous polymer matrix of the particles used for the SPE may be functionalized with a hydroxy group (—OH), in particular an —OH. Non-limiting examples for such magnetic particles are described in WO2018189286A1. A non-limiting example for such magnetic particles are the beads of type A as used in the appended examples.

In embodiments, the porous polymer matrix may comprises nitrogen atoms, in particular at least one positively charged nitrogen atom. In particular embodiments, the porous matrix of the magnetic beads may comprise two positively charged nitrogen atoms. Positively charged preferably refers to permanently positively charged.

Non-limiting examples for such particles (in particular magnetic particles) are described in WO2019141779A1. A non-limiting example for such magnetic particles are the beads of type B as used in the appended examples. Without being bound by theory, the appended examples indicate that the nitrogen atoms, in particular positively charged nitrogen atoms in the polymer matrix may facilitate capturing and thus recovery efficiency for many steroids, including 11-oxygenated steroids. Moreover, without being bound by theory, the appended Examples support that DHEAS is adsorbed with lower efficiency. However, this can be advantageous because DHEAS is typically present at much higher concentrations in samples (e.g. serum or plasma) than the other steroids detected. Without a reduced recovery, the DHEAS signal may overlap with the signals of other steroids to a certain extent. A higher DHEAS signal may cause saturation of the detector or suppress signals of other steroids to a certain extent.

In embodiments, a particle may be hypercrosslinked magnetic particle, wherein the porous polymer is hypercrosslinked. In particular, the porous polymer may be hypercrosslinked in a form obtained or obtainable by a Friedel-Crafts reaction.

In a preferred embodiment, the hypercrosslinking may be achieved with a hypercrosslinking bond, which comprises at least two nitrogen atoms, in particular wherein the hypercrosslinking bond comprises a diamine. Such a particle (e.g. magnetic particle) will be a porous polymer matrix that comprises nitrogen atoms, as described above. Again, it is particularly preferred that the hypercrosslinking bond comprises at least one (e.g. two) positively charged nitrogen atom.

In embodiments, a hypercrosslinking bond that consists of a molecule comprising at least two nitrogen atoms within its structure which are part of the hypercrosslinking bond, wherein the molecule comprising at least two nitrogen atoms within its structure has the general structure of formula 1

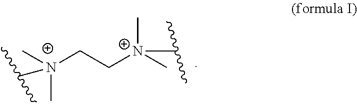 [SMILES: *[N+](C)(C)CC[N+](*)(C)C]

The beads of type B are non-limiting examples comprising hyper crosslinks with formula 1.

Solid phase extraction in the context of the invention may include flow through based solid phase extraction and batch-type solid phase extraction.

Flow through based solid phase extraction means that the solid phase is retained in a container (e.g. a cartridge) and the sample (or a pretreated sample) is applied to the solid phase in a flow through process. Optionally, a flow through may include a defined incubation time in which the sample is contacted with the solid phase while the flow through is blocked. For example, flow through based solid phase extraction maybe conducted with solid phase extraction cartridge/columns or a solid phase tips. A flow through based solid phase extraction may comprise one or more wash steps in which residual liquid phase is removed.

“Batch-type based solid phase extraction”, as used herein, refers to solid phase based separation methods that do not include a flow through step. “Batch-type based solid phase extraction” includes: bringing the sample (or pretreated sample) into contact with the solid phase and optionally incubating the sample in presence of the solid phase for a defined time (required for analyte binding or binding of one or more other sample constituents depending on the separation principle); and separating the solid phase from the liquid phase by means different from flow through (e.g. pelleting). In particular, batch-type solid phase extraction includes an embodiment in which the solid phase is formed by beads (e.g. microbeads and in particular magnetic beads) and wherein the separation of the solid phase from the liquid phase during SPE is achieved by pelleting the beads. Pelleting the beads may be achieved by centrifugation or other means. In specific embodiments, pelleting the beads may not involve centrifugation. In a particular embodiment, beads may be magnetic and the beads may be pelleted by magnetic force.

In a particular embodiment, the solid phase used in the SPE may be a batch-type SPE, preferably a batch type SPE using (micro)beads, even more preferably a batch type SPE using magnetic (micro)beads. The batch-type SPE may be based on binding/capturing the one or more steroid analytes to the solid phase used in the SPE. In other embodiments, other sample constituents may bind to the solid phase and the one or more steroids may remain in the liquid phase.

The final solution comprising the analytes to be detected and/or quantified (i.e. the one or more steroids) obtained by SPE is referred to herein as “SPE extract” Depending on the separation principle employed in the SPE (i.e. retardation of the analyte(s) on the solid phase or in the liquid phase), the “SPE extract” may correspond to the liquid phase of the sample obtained after incubation with the solid phase (since in these embodiments analyte(s) do not bind to solid phase) or may correspond to the eluate obtained by elution from the solid phase using an elution solvent/solution (in these embodiments the analytes are bound to the solid phase and subsequently eluted). In particular embodiments, the one or more steroids may be retarded on the solid phase and the SPE extract may correspond to eluate obtained by elution from the solid phase using an elution solvent.

In particular embodiments, the SPE may comprise
a) binding/capturing the one or more steroids to a solid phase;
b) optionally one or more wash steps; and
c) eluting the one or more steroids from the solid phase, wherein the eluate obtained is referred to as SPE extract and comprises the one or more steroids.

The binding of the one or more steroids to the solid phase may involve incubation of the solid phase with the sample for a predefined time under conditions that allow capturing the one or more steroids to be detected. In the context of the invention the incubation is preferably for 4 to 15 min, in particular 9 min. Keeping the incubation time short has the advantage that the sample preparation time for the mass spectrometry analysis is reduced which is of high importance especially in an automated mass spectrometry analyzer system. Exemplary solid phases, which allow such short incubation time, are disclosed herein above.

The incubation of the solid phase with the sample may be conducted at a temperature of 25 to 45° C., in particular 35 to 40° C., in particular 37° C.

In particular embodiments, the SPE may be a magnetic bead based workflow. The magnetic bead based workflow may comprise:
a) binding/capturing the one or more steroids to magnetic microbeads;
b) optionally one or more wash steps; and
c) eluting the one or more steroids from the solid phase, wherein the eluate obtained is referred to as SPE extract and comprises the one or more steroids.

The magnetic bead based workflow may further comprise:
d) separating the SPE extract from the beads.

This separating may be achieved by pelleting the magnetic beads (e.g. by magnetic force) and removing the eluate.

Accordingly, in a particular embodiment the present invention provides for a method for detecting or quantifying one or more steroids in a sample using mass spectrometry, wherein said method comprises
a) extracting said one or more steroids from the sample using a magnetic bead based workflow (e.g. as described elsewhere herein),
b) concentrating the one or more steroids, said concentrating comprising evaporating solvent from the extract obtained in a), and
c) detecting and/or quantifying the one or more steroids in the sample using mass spectrometry.

In other words, “extracting the one or more steroids from the sample using solid phase extraction (SPE) so as to obtain an SPE extract comprising the one or more steroids” may in embodiments of the invention be “extracting said one or more steroids from the sample by a magnetic bead based workflow (e.g. as described elsewhere herein)”.

A “magnetic bead workflow” refers to a method in which magnetic beads (e.g. magnetic microbeads) are used to extract the one or more steroids from the sample. The magnetic beads may bind the one or more steroids while other sample constituents may partially or fully removed with the liquid phase. Alternatively, the magnetic beads may bind one or more of the sample constituents and the one or more steroids may remain in solution.

The SPE of the method of the invention may comprise one or more wash steps using a wash solution. The method may, for example, comprise one or two wash steps using a wash solution. The wash steps may be conducted after binding/capturing the one or more steroids to the solid phase and prior to eluting the one or more steroids from the solid phase.

In embodiments, the wash solution used for the one or more wash steps in the SPE may have a pH of 2 to 4, in particular 2.5 to 3.5 (e.g. 3) and in particular 2.6. The appended Examples demonstrate that using a wash buffer having such acidic pH range increases recovery of steroids, in particular one or more 11-oxygenated steroids, by the sample preparation. The pH1 of the wash solution may be adjusted using formic acid. For instance, a final concentration 40 mM formic acid may be used. In embodiments, the wash solution may comprise an organic solvent such as methanol, ACN or DMSO at a concentration that does not elute the one or more steroids from the solid phase. Exemplary, the wash solution may be aqueous (e.g. water) with an organic content of 0-10 vol %, 3-8 vol % or 5 vol %.

In embodiments, the SPE may be a batch-type SPE and the one or more wash steps may comprise
i) adding the wash buffer to the solid phase;
ii) incubating the sample with the wash buffer, in particular for 40 to 80 sec, in particular 60 to 70 sec, in particular 65 sec;
iii) pelleting the solid phase (e.g. by magnetic force), and
iv) removing the supernatant.

The elution of the one or more steroids from the solid phase may be achieved with an elution solvent. The elution solvent may be added to the solid phase and the solid phase may be incubated in the presence of the elution solvent for a predefined time (e.g. 275 sec). The incubation of the solid phase in presence of the elution solvent may be conducted for 30 to 400 sec, in particular 50 to 150 sec, in particular 108 sec. The time may be adjusted depending on which solid phase is used and depending on how harsh the elution solvent is.

The composition of the elution solvent can be selected dependent on the solid phase, analyte and the interaction principle between the analyte and the solid phase. For instance, the elution solvent may comprise acetonitrile (ACN) in particular at a concentration of 40-100 vol %, in particular 45-90 vol %, in particular 50-70 vol %, in particular 60 vol %. The aforementioned ACN based elution solvents may especially be employed in embodiments using particles, such as the magnetic particles disclosed herein elsewhere.

In embodiments, the elution solvent may comprises methanol, in particular at a concentration of 70-100 vol %, in particular 80-90 vol %, in particular 80 vol %. The elution solvent may be a mixture of methanol and water.

The volume of the elution solvent added to the solid phase (e.g. beads) may be 30 to 150%, in particular 100 to 130% and even more particularly 100-120% of the sample volume subjected to SPE. In one embodiment, a sample volume of 150 μl (e.g. 150 μl of a blood sample such as serum or plasma) may be employed for SPE and the elution volume may be 180 μl.

The method of the invention may comprise using one or more internal standards (ISTD), in particular isotope labeled internal standards. ISTDs may be used for quantification of the analytes. For example, one internal standard may be added for each steroid to be detected and/or quantified. In embodiments, less internal standards than the number of steroids to be analyzed may be added. In these embodiments, certain internal standards may serve as internal standard for more than one of the steroids to be detected (e.g. two). The ISTDs in this case may be selected such they are physicochemical similar to all steroids for which they serve as ISTD. The one or more internal standards are preferably added to the sample in a predefined and known amount prior to SPE and the optional pretreatment step.

An “internal standard (ISTD)” is typically a compound that exhibits similar physicochemical properties as the analyte of interest when subjected to the mass spectrometric detection workflow (i.e. including any pre-treatment, enrichment and actual detection step). Moreover, an ISTD is typically selected such that it does not naturally occur in the samples to be measured in significant amounts (e.g. 1% or less of the amounts of the corresponding steroid to be detected). For example, an ISTD may be an isotope labelled steroid, such as the steroid to be detected. Although the ISTD exhibits similar properties as the analyte of interest, it is still clearly distinguishable from the analyte of interest. Exemplified, during chromatographic separation, such as gas or liquid chromatography, the ISTD has about the same retention time as the analyte of interest front the sample. Thus, both the analyte and the ISTD enter the mass spectrometer at the same time. The ISTD however, exhibits a different molecular mass than the analyte of interest from the sample. This allows a mass spectrometric distinction between ions from the ISTD and ions from the analyte by means of their different mass/charge (m/z) ratios. Both are subject to fragmentation and provide daughter ions. These daughter ions can be distinguished by means of their m/z ratios from each other and from the respective parent ions. Consequently, a separate determination and quantification of the signals from the ISTD and the analyte can be performed. Since the ISTD has been added in known amounts, the signal intensity of the analyte from the sample can be attributed to a specific quantitative amount of the analyte. Thus, the addition of an ISTD allows for a relative comparison of the amount of analyte detected, and enables unambiguous identification and quantification of the analyte(s) of interest present in the sample when the analyte(s) reach the mass spectrometer. Typically, but not necessarily, the ISTD is an isotopically labelled variant (comprising e.g. at least three of ²H, ¹³C, and/or ¹⁵N etc. label) of the analyte of interest.

Exemplary substances that can be used as ISTDs for the one or more steroids to be detected by the method of the invention are listed herein below in Table 2, below. These compounds are commercially available and/or known in the art.

The method of the invention involves the step of concentrating the one or more steroids, said concentrating comprising evaporating the solvent from the SPE-extract.

“Concentrating the one or more steroids” means that a solution with a concentration of the one or more steroids that is higher than the concentration of the one or more steroids in the SPE extract is generated.

“Evaporating the solvent” means that liquid is evaporated in a manner that the one or more steroids do not evaporate.

The step of “concentrating the one or more steroids” may in particular involve evaporation of the SPE extract to dryness and subsequent resuspension of the residual in a diluent (also referred to as reconstitution solution herein), wherein the volume of the diluent is smaller than the volume of the SPE extract and/or sample. For instance, the diluent volume in which the dried residual of the SPE extract after evaporation is re-suspended may corresponds to 10-50%, in particular 20-40%, in particular 24-28%, in particular 26.7% of the volume of the sample subjected to SPE. In one embodiment, a sample volume of 150 μl may be subjected to a magnetic bead based SPE workflow, the one or more steroids may be eluted in the beads in a volume of 180 μl (SPE extract), the eluate (SPE extract) may be evaporated to dryness and the residual may be re-suspended in 40 μl.

In embodiments, the evaporation may be conducted such that the solvent of the SPE extract is not evaporated fully. The evaporation may be conducted such that a liquid volume reduction of 50 to 100%, in particular 60 to 85% is achieved. The concentrated SPE extract resulting from such incomplete evaporation may either be directly used for LC or may be diluted with a diluent. The partial evaporation may increase analyte concentration and reduce organic content. Dilution may further adjust the organic contents to levels that are better compatible with LC and thus lead to a better analyte separation during LC.

A suitable diluent can resolve the dried residuals of the evaporation and/or dilute a concentrated SPE extract such that it does not interfere with the downstream mass spectrometry analysis (in particular LC). A non-limiting example for a suitable reconstitution solution is a 10-30% methanol solution or water.

Concentrating the one or more steroids in the SPE extract may preferably achieved in less than 12 min, in particular in less than 10 min.

It has been observed by the inventors that adjusting the organic solvent (e.g. methanol or acetonitril) content to 10-30 vol %, in particular 20-30 vol % is advantageous for achieving efficient separation in liquid chromatography. Thus, by combining evaporation and optionally dilution such that the organic solvent content is reduced and at the same time the total volume is reduced compared to the initial sample volume. This is desired to increase LC performance and to increase analyte concentration, respectively.

The concentration step has the advantage that the concentration(s) of the one or more steroid(s) are increased for the mass spectrometry analysis such that recovery of the one or more steroids increases and consequently also the lower limits of quantification can be reached. Especially by combining SPE and concentration a signal increase relative to the background signal could be achieved. The increase in the concentrations of the one or more steroids by the concentration step also allows using lower sample volumes for a given limit of quantification and increases the amount of analyte subjected to mass spectrometry in a defined volume. Moreover, steroid concentration using evaporation of solvent has the advantage that volatile liquids such as organic solvents comprised in the SPE extract (e.g. ACN or methanol used for eluting the one or more steroids from the solid phase) may be removed. Such solvents can interfere with the liquid chromatography of a LC comprising mass spectrometry workflow.

Evaporation may be achieved with different evaporation systems or chambers known in the art. The evaporation system or chamber may be part of the mass spectrometry system. The evaporation may be conducted fully automated, i.e. without manual handling steps. In embodiments, an evaporation system that does not use centrifugation may be employed. In a particular embodiment, the evaporation of the SPE solvent (and optionally the addition of diluent) may be achieved in less than 12 min, in particular less than 10 min.

As mentioned above, the mass spectrometry analysis may involve liquid chromatography (LC). In particularly preferred embodiments, the mass spectrometry analysis may be LC-MS or LC-MS-MS.

In embodiments, liquid chromatography may be high-pressure liquid chromatography (HPLC). The HPLC may be based on different column materials known in the art. The flow rate of the HPLC can be adapted depending on the steroid analytes and needs. In specific embodiments, the flow rate of the HPLC may be 0.5-1.5 mil/min, in particular 1.0 ml/min.

In some embodiments, the liquid chromatography of the mass spectrometry analysis may be rapid LC.

In embodiments, the HPLC separation principle may be a reversed phase HPLC (RP-HPLC) RP-HPLC may be but is not limited to a C18-HPLC. RP-HPLC may use a gradient of methanol (e.g. 50 vol % to 75 vol %) as mobile phase. Optionally the mobile phase may comprise 0.1 vol % formic acid. The mobile phase gradient (also referred to as elution gradient) may be linear or non-linear. In a particular embodiment, the mobile phase gradient is linear.

The elution gradient may be established by mixing a solvent A being aqueous solution optionally comprising 0.1 vol % formic acid and a solvent B being methanol optionally comprising 0.1 vol % formic acid.

In embodiments, the mobile phase gradient (e.g. linear gradient) may be configured such that the one or more steroids are separated within 1 to 2 min, in particular 1.1 to 1.5 min and in particular 1.2 min.

In embodiments, the LC settings may be configured such that the one or more 11-oxygenated C19 steroids elute from the LC in a first time period and the other steroids (e.g. classical steroids) elute from the LC in a second period. The mass spectrometry measurement times and settings (e.g. MRM settings) assigned to the 11-oxygenated C19 steroids and the other steroids (e.g. classical steroids) may be assigned accordingly. For instance, if a LC gradient as defined in the appended example 2 is employed the time between 0.55 and 0.70 min may be used for the detection and/or quantification of 11-oxygenated steroids and the time between 0.70 min and 1.2 min may be used for detection of other steroids (e.g. classical steroids). The appended Examples demonstrate that combining (i) SPE of the one or more steroids and (ii) concentrating the one or more steroids using evaporation improves the limit of detection for steroids and in particular 11-oxygenated C19 steroids. The combination of these steps surprisingly increased the peak signal of the steroids while background signals did not increase to a similar extent, i.e. no matrix effect was observed. Thus, the signal to noise ratio (e.g. for the ions derived from 11-oxygenated steroids) can be improved by combining both steps which particularly enables reliable detection and/or quantification of the one or more steroids from low sample volumes (e.g. 15-500 μl). Another advantage of introducing two measurement periods is that this setting allows using positive and negative mode in the two periods, respectively. This increases sensitivity because using positive and negative recording modes simultaneously dampens sensitivity.

Ionization in the mass spectrometry analysis may be based on different techniques as described elsewhere herein. In one embodiment, electrospray ionization (ESI).

The MS device used for the mass spectrometry analysis may be a tandem mass spectrometer, in particular a triple quadrupole device.

In a particular embodiment, the method of the invention may be automated “Automated” means that except for the step of applying the sample and reagents to the system one or less, preferably no manual handling steps are required. Manual handling steps include in particular the manual addition of a reagent to the sample and the transfer of the sample during processing from one device to another.

In embodiments, the method of the invention may not comprise a centrifugation step. In particular, the SPE and/or the concentration may be conducted without centrifugation. Preventing a centrifugation step (e.g. by using a magnetic bead based SPE workflow) has the advantage that it can be automated easier and that a sample preparation system does not require a centrifuge.

In embodiments, the method of the invention may not comprise liquid-liquid extraction, in particular not in the sample preparation. In other words, the SPE and the concentration steps may be the only sample preparation steps before mass spectrometry (e.g. LC-MS, in particular LC-MS/MS). As demonstrated by the appended. Examples, sample preparation does not require liquid-liquid extraction steps, which are typically cumbersome and consume organic solvents.

The method of the invention may in particular be performed in a random-access compatible mode and using a random access compatible system “Random-access” preferably means that the reagents and system settings of the described invention are compatible with other assays addressing different steroids or non-steroids without the need of system adaptation or equilibration, in particular manual system adaptation or equilibration (including changes to the mass spectrometry and/or LC settings).

The mass spectrometry analysis of the method of the invention may be conducted using a Multiple Reaction Monitoring (MRM) mode.

In embodiments of the method of the present disclosure, steroids may comprise one or more 11-oxygenated steroids and one or more other steroids. The MS analysis may be performed two periodic such that the parent ion(s) and/or fragment ion(s) of the one or more 11-oxygenated steroids are detected in a first period and that the parent ion(s) and/or fragment ion(s) of the one or more other steroids are detected in a second period. For example, the cycle time of the first period may be from 120 ms to 200 ms (e.g. 130 ms, 140 ms, 150 ms, 160 ms, 170 ns, 180 ms or 190 ms) or preferably 170 ms. The cycle time of the second period may be from 150 to 270 ms (e.g. 160 ms, 170 ms, 180 ms, 190 ms, 200 ins, 210 ms, 220 ms, 230 ms, 240 ms or 250 ms) or preferably 230 ms, To achieve this LC of a LC comprising MS analysis may be configured such that the one or more 11-oxygenated steroids elute in a first period and the other steroids elute in a second period. In embodiments the LC settings described herein above may be employed. Dwell times can be prolonged by a two periodic measurement compared to a one periodic measurement. As demonstrated by the appended example, a two periodic setting can increase sensitivity in the detection of 11-oxygenated steroids.

In embodiments, for each of the one or more steroids a parent ion may be generated and two fragment ions thereof are generated and detected in the MS analysis (e.g. an LC-MS/MS). One of the two fragment ions may be used for quantification and the second fragment ion may be used as qualifier (i.e. be used as an identifier for the presence of a respective steroid).

In the mass spectrometry analysis a parent ion for each of the one or more steroids may be generated. A parent ion may, for example be a [M+H]⁻ ion (positive mode) or a [M−H]⁻ ion (negative mode). Also less preferred, also water loss ions may be generated and used as parent ion for fragmentation. The parent ion may be selected for fragmentation to generate one or more fragment ions (also referred to as daughter ion(s) herein). The fragment ion(s) may be used for detection and/or quantification of the one or more steroids. In embodiments, the mass spectrometry analysis may be a tandem NIS (MS/MS) analysis in which from each of the one or more steroids a parent ion is generated and selected for fragmentation, wherein the fragmentation is configured to generate at least two fragment ions per parent ion. In embodiments, one fragment ion may be used for detection. In embodiments, two fragment ions may be used for detection. In embodiments, one fragment ion may be used for quantification. In embodiments, two fragment ions may be used for quantification.

In embodiments, the one or more steroids may comprise 11OHA4. For 11OHA4, a parent ion having an m/z value of 303.1±0.5 may be generated and selected for fragmentation. The parent ion is preferably positively charged. In embodiments, the parent ion may be fragmented in a first fragment ion having an m/z ratio of 267.2±0.5 and/or a second fragment ion having an m/z ratio of 121.1±0.5. The first and second fragment ions are preferably positively charged. The first and/or second fragment ions may be used for detection and/or quantification. Particularly, the first 11OHA4-fragment ion with an m/z ration of 267.2±0.5 may be used for quantification of 11OHA4. The second 11OHA4-fragment ion having an m/z ratio of 121.1±0.5 may be used for detection (i.e. identification).

An exemplary but non-limiting example for an ISTD for quantifying 11OHA4 is an isotope-labeled 11OHA4, in particular a deuterated 11OHA4 and even more particularly 11OHA4-d4 (9, 11, 12, 12-d4). In the embodiments using 11OHA4-d4 (9, 11, 12, 12-d4) as an ISTD for 11 OHA4, a parent ion having an m/z ratio of 307.1 may be generated. The parent ion is preferably positively charged. The parent ion may be fragmented into a first fragment ion having an m/z ratio of 121.1±0.5 and/or a second fragment ion having an m/z ratio of 148.2±0.5. The first and second fragment ions are preferably positively charged. In particular embodiments, the first fragment ion having an m/z ratio of 121.1±0.5 may be used for quantification. In embodiments, the second fragment ion having an m/z ratio of 148.2±0.5 may be used as identifier. In embodiments, the one or more steroids may comprise 11KT. For 11KT, a parent ion having an m/z value of 303.1±0.5 may be generated and selected for fragmentation. The parent ion is preferably positively charged. In embodiments, the parent ion may be fragmented in a first fragment ion having an m/z ratio of 121.1±0.5 and/or a second fragment ion having an m/z ratio of 259.2±0.5. The first and second fragment ions are preferably positively charged. The first and/or second fragment ions may be used for detection and/or quantification. Particularly, the first 11KT-fragment ion with an m/z ration of 121.1±0.5 may be used for quantification of 11KT. The second 11KT-fragment ion having an m/z ratio of 259.2±0.5 may be used for detection (i.e. identification).

An exemplary but non-limiting example for an ISTD for quantifying 11KT is an isotope-labeled 11KT, in particular a deuterated 11KT and even more particularly 11KT-d₃ (16,16,17-d3). In the embodiments using 11KT-d₃ (16,16,17-d3) as an ISTD for 11KT, a parent ion having an m/z ratio of 306.1 may be generated. The parent ion is preferably positively charged. The parent ion may be fragmented into a first fragment ion having an m/z ratio of 262.2±0.5 and/or a second fragment ion having an m/z ratio of 121.1±0.5. The first and second fragment ions are preferably positively charged. In particular embodiments, the first fragment ion having an m/z ratio of 262.2±0.5 may be used for quantification. In embodiments, the second fragment ion having an m/z ratio of 121.1±0.5 may be used as identifier.

In embodiments, the one or more steroids may comprise 11KA. For 11KA, a parent ion having an m/z value of 301.05±0.50 may be generated and selected for fragmentation. The parent ion is preferably positively charged. In embodiments, the parent ion may be fragmented in a first fragment ion having an m/z ratio of 257.2±0.5 and/or a second fragment ion having an m/z ratio of 121.2±0.5. The first and second fragment ions are preferably positively charged. The first and/or second fragment ions may be used for detection and/or quantification. Particularly, the first 11KA-fragment ion with an m/z ration of 257.2±0.5 may be used for quantification of 11KA. The second 11KA-fragment ion having an m/z ratio of 121.2±0.5 may be used for detection (i.e. identification).

An exemplary but non-limiting example for an ISTD for quantifying 11KA is an isotope-labeled 11KA, in particular a deuterated 11KA. In embodiments, due to the chemical similarity also deuterated 11KT such as 11KT-d₃ (16,16,17-d3) may be used as an ISTD for 11KA (see appended examples). For 11KT-d₃ (16,16,17-d3) a parent ion having an m/z ratio of 306.1 may be generated. The parent ion is preferably positively charged. The parent ion may be fragmented into a first fragment ion having an m/z ratio of 262.2±0.5 and/or a second fragment ion having an m/z ratio of 121.1±0.5. The first and second fragment ions are preferably positively charged. In particular embodiments, the first fragment ion having an m/z ratio of 262.2±0.5 may be used for quantification. In embodiments, the second fragment ion having an m/z ratio of 121.1±0.5 may be used as identifier.

In embodiments, the one or more steroids may comprise 11OHT. For 11OHT, a parent ion having an m/z value of 305.2±0.5 may be generated and selected for fragmentation. The parent ion is preferably positively charged. In embodiments, the parent ion may be fragmented in a first fragment ion having an m/z ratio of 269.2±0.5 and/or a second fragment ion having an m/z ratio of 121.1±0.5. The first and second fragment ions are preferably positively charged. The first and/or second fragment ions may be used for detection and/or quantification. Particularly, the first 11OHT-fragment ion with an m/z ration of 269.2±0.5 may be used for quantification of 11OHT. The second 11OHT-fragment ion having an m/z ratio of 121.1±0.5 may be used for detection (i.e. identification).

An exemplary but non-limiting example for an ISTD for quantifying 11OHT is an isotope-labeled 11OHT (e.g. deuterated 11OHT). In embodiments, due to the chemical similarity also isotope-labeled 11OHA4, in particular a deuterated 11OHA4 and even more particularly 11OHA4-d4 (9, 11, 12, 12-d4) may be employed as ISTD for 11OHT (see appended examples). In the embodiments using 11OHA4-d4 (9, 11, 12, 12-d4) as an ISTD for 11OHT, a parent ion having an m/z ratio of 307.1 may be generated. The parent ion is preferably positively charged. The parent ion may be fragmented into a first fragment ion having an m/z ratio of 121.1±0.5 and/or a second fragment ion having an m/z ratio of 148.2±0.5. The first and second fragment ions are preferably positively charged. In particular embodiments, the first fragment ion having an m/z ratio of 121.1±0.5 may be used for quantification. In embodiments, the second fragment ion having an m/z ratio of 148.2±0.5 may be used as identifier.

In embodiments, the one or more steroids may comprise two or more (e.g. 2, 3 or 4) 11-oxygenated C19 steroids selected from 11OHA4, 11KT, 11KA and 11OHT and the above mentioned parent and fragment ions may be generated and used accordingly. Optionally also combinations of the above mentioned preferred ISTDs may be employed.

In embodiments, the one or more steroids may comprise T. For T, a parent ion having an m/z value of 289.2±0.5 may be generated and selected for fragmentation. The parent ion is preferably positively charged. In embodiments, the parent ion may be fragmented in a first fragment ion having an m/z ratio of 97.1±0.5 and/or a second fragment ion having an m/z ratio of 109.1±0.5 The first and second fragment ions are preferably positively charged. The first and/or second fragment ions may be used for detection and/or quantification. Particularly, the first T-fragment ion with an m/z ration of 97.1±0.5 may be used for quantification of T. The second T-fragment ion having an m/z ratio of 109.1±0.5 may be used for detection (i.e. identification).

An exemplary but non-limiting example for an ISTD for quantifying T is an isotope-labeled T, in particular a T comprising a C¹³ and even more particularly T-¹³C₃ (a testosterone comprising three ¹³C). In the embodiments using T-¹³C₃ as an ISTD for T, a parent ion having an m/z ratio of 292.2 may be generated. The parent ion is preferably positively charged. The parent ion may be fragmented into a first fragment ion having an m/z ratio of 100.0±0.5 and/or a second fragment ion having an m/z ratio of 112.1±0.5. The first and second fragment ions are preferably positively charged. In particular embodiments, the first fragment ion having an m/z ratio of 100.0±0.5 may be used for quantification. In embodiments, the second fragment ion having an m/z ratio of 112.1±0.5 may be used as identifier.

In embodiments, the one or more steroids may comprise A4. For A4, a parent ion having an m/z value of 287.2±0.5 may be generated and selected for fragmentation. The parent ion is preferably positively charged. In embodiments, the parent ion may be fragmented in a first fragment ion having an m/z ratio of 97.1±0.5 and/or a second fragment ion having an m/z ratio of 109.1±0.5. The first and second fragment ions are preferably positively charged. The first and/or second fragment ions may be used for detection and/or quantification. Particularly, the first A4-fragment ion with an m/z ration of 97.1±0.5 may be used for quantification of A4. The second A4-fragment ion having an m/z ratio of 109.1±0.5 may be used for detection (i.e. identification).

An exemplary but non-limiting example for an ISTD for quantifying A4 is an isotope-labeled A4, in particular an A4 comprising a C¹³ and even more particularly A4-¹³C₃ (an A4 comprising three ¹³C). In the embodiments using A4-¹³C₃ as an ISTD for A4, a parent ion having an m/z ratio of 292.2 may be generated. The parent ion is preferably positively charged. The parent ion may be fragmented into a first fragment ion having an m/z ratio of 100.0±0.5 and/or a second fragment ion having an m/z ratio of 112.1±0.5. The first and second fragment ions are preferably positively charged. In particular embodiments, the first fragment ion having an m/z ratio of 100.0±0.5 may be used for quantification. In embodiments, the second fragment ion having an m/z ratio of 112.1±0.5 may be used as identifier.

In embodiments, the one or more steroids may comprise DHEA. For DHEA, a parent ion having an m/z value of 289.2±0.5 may be generated and selected for fragmentation. The parent ion is preferably positively charged. In embodiments, the parent ion may be fragmented in a first fragment ion having an m/z ratio of 213.1±0.5 and/or a second fragment ion having an m/z ratio of 91.0±0.5. The first and second fragment ions are preferably positively charged. The first and/or second fragment ions may be used for detection and/or quantification. Particularly, the first DHEA-fragment ion with an m/z ration of 213.1±0.5 may be used for quantification of DHEA. The second DHEA-fragment ion having an m/z ratio of 91.0±0.5 may be used for detection (i.e. identification).

An exemplary but non-limiting example for an ISTD for quantifying DHEA is an isotope-labeled DHEA, in particular a DHEA comprising a C¹³ and even more particularly DHEA-¹³C₃ (a DHEA comprising three ¹³C). In the embodiments using DHEA-¹³C₃ as an ISTD for DHEA, a parent ion having an m/z ratio of 292.2 may be generated. The parent ion is preferably positively charged. The parent ion may be fragmented into a first fragment ion having an m/z ratio of 256.2±0.5 and/or a second fragment ion having an m/z ratio of 216.4±0.5 The first and second fragment ions are preferably positively charged. In particular embodiments, the first fragment ion having an m/z ratio of 256.2±0.5 may be used for quantification. In embodiments, the second fragment ion having an m/z ratio of 216.4±0.5 may be used as identifier.

In embodiments, the one or more steroids may comprise DHEAS. For DHEAS, a parent ion having an m/z value of 367.2±0.5 may be generated and selected for fragmentation. The parent ion is negatively charged. In embodiments, the parent ion may be fragmented in a first fragment ion having an m/z ratio of 80.1±0.5 and/or a second fragment ion having an m/z ratio of 97.1±0.5 The first and second fragment ions are preferably negatively charged. The first and/or second fragment ions may be used for detection and/or quantification. Particularly, the first DHEAS-fragment ion with an m/z ration of 80.1±0.5 may be used for quantification of DHEAS. The second DHEAS-fragment ion having an m/z ratio of 97.1±0.5 may be used for detection (i.e. identification).

An exemplary but non-limiting example for an ISTD for quantifying DHEAs is an isotope-labeled DHEAS, in particular a DHEAS comprising a C¹³ and even more particularly DHEAS-¹³C; (an DHEAS comprising three ¹³C). In the embodiments using DHEAS-¹³C₃ as an ISTD for DHEAS, a parent ion having an m/z ratio of 370.2 may be generated. The parent ion is negatively charged. The parent ion may be fragmented into a first fragment ion having an miz ratio of 97.0±0.5 and/or a second fragment ion having an m/z ratio of 80.0±0.5. The first and second fragment ions are preferably negatively charged. In particular embodiments, the first DHEAS-¹³C₃ fragment ion having an m/z ratio of 97.0±0.5 may be used for quantification. In embodiments, the second DHEAS-¹³C₃ fragment ion having an m/z ratio of 80.0±0.5 may be used as identifier.

In embodiments, the one or more steroids may comprise ET. For ET, a parent ion having an m/z value of 289.2±0.5 may be generated and selected for fragmentation. The parent ion is preferably positively charged. In embodiments, the parent ion may be fragmented in a first fragment ion having an m/z ratio of 109.0±0.5 and/or a second fragment ion having an m/z ratio of 97.0±0.5. The first and second fragment ions are preferably positively charged. The first and/or second fragment ions may be used for detection and/or quantification. Particularly, the first ET-fragment ion with an m/z ration of 109.0±0.5 may be used for quantification of ET. The second ET-fragment ion having an m/z ratio of 97.0±0.5 may be used for detection (i.e. identification).

An exemplary but non-limiting example for an ISTD for quantifying ET is an isotope-labeled ET, in particular an ET comprising a C¹³ and even more particularly ET-¹³C₃ (an ET comprising three ¹³C). In the embodiments using ET-¹³C as an ISTD for ET, a parent ion having an m/z ratio of 292.2 may be generated. The parent ion is preferably positively charged. The parent ion may be fragmented into a first fragment ion having an m/z ratio of 100.1±0.5 and/or a second fragment ion having an m/z ratio of 112.0±0.5. The first and second fragment ions are preferably positively charged. In particular embodiments, the first fragment ion having an m/z ratio of 100.1±0.5 may be used for quantification. In embodiments, the second fragment ion having an m/z ratio of 112.0±0.5 may be used as identifier.

The present invention relates to a method for detecting and quantifying one or more steroids (preferably comprising at least one 11-oxygenated C19 steroid). As described in detail herein elsewhere, the method of the invention comprises a specific combination of sample preparation steps. i.e. SPE extraction of the one or more steroids and a concentration step using solvent evaporation.

Thus, in one aspect the present invention also provides for a method for preparing a sample for a mass spectrometry analysis (e.g. LC-MS/MS) detecting one or more steroids, said method comprising:
a) extracting the one or more steroids from the sample using solid phase extraction (SPE) so as to obtain an SPE extract comprising the one or more steroids;
b) concentrating the one or more steroids, said concentrating comprising evaporating solvent from the SPE-extract obtained in a).

What has been said above with respect to the method for detecting and quantifying one or more steroids (preferably comprising at least one 11-oxygenated C19 steroid) applies mutatis mutandis to the method for preparing a sample for a mass spectrometry analysis (e.g. LC-MS/MS) detecting one or more steroids.

The term “mass spectrometry” (“Mass Spec” or “MS”) relates to an analytical technology used to identify compounds by their mass. MS is a method of filtering, detecting, and measuring ions based on their mass-to-charge ratio, or “m/z”. NIS technology generally includes (1) ionizing compounds to form charged compounds; and (2) detecting the molecular weight of the charged compounds and calculating a mass-to-charge ratio. The compounds may be ionized and detected by any suitable means. A “mass spectrometer” generally includes an ionizer and an ion detector. In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). The term “ionization” or “ionizing” refers to the process of generating an analyte ion having a net electrical charge equal to one or more electron units. Negative ions are those having a net negative charge of one or more electron units, while positive ions are those having a net positive charge of one or more electron units. The NIS method may be performed either in “negative ion mode”, wherein negative ions are generated and detected, or in “positive ion mode” wherein positive ions are generated and detected.

“Tandem mass spectrometry” or “MS/MS” involves multiple steps of mass spectrometry selection and detection, wherein fragmentation of the analyte occurs in between the steps. In a tandem mass spectrometer, ions are formed in the ion source and separated by mass-to-charge ratio in the first stage of mass spectrometry (MS1). Ions of a particular mass-to-charge ratio (precursor ions or parent ions) are selected and fragment ions (also referred to as daughter ions) are created by collision-induced dissociation, ion-molecule reaction, and/or photodissociation. The resulting ions are then separated and detected in a second stage of mass spectrometry (MS2).

Typically, for the mass spectrometry measurement, the following three steps are performed:
(1.) a sample comprising an analyte of interest is ionized, usually by adduct formation with cations, often by protonation to cations. Ionization sources include but are not limited to electrospray ionization (ESI) and atmospheric pressure chemical ionization (APCI)
(2.) the ions are sorted and separated according to their mass and charge. High-field asymmetric-waveform ion-mobility spectrometry (FAIMS) may be used as ion filter
(3.) the separated ions are then detected, e.g. in multiple reaction mode (MRM), and the results are displayed on a chart.

The term “electrospray ionization” or “ESI.” refers to methods in which a solution is passed along a short length of capillary tube, to the end of which is applied a high positive or negative electric potential. A solution reaching the end of the tube is is vaporized (nebulized) into a jet or spray of very small droplets of solution in solvent vapour. This mist of droplets flows through an evaporation chamber, which is heated slightly to prevent condensation and to evaporate solvent. As the droplets get smaller the electrical surface charge density increases until such time that the natural repulsion between like charges causes ions as well as neutral molecules to be released.

The term “atmospheric pressure chemical ionization” or “APCI,” refers to mass spectrometry methods that are similar to ESI; however, APCI produces ions by ion-molecule reactions that occur within a plasma at atmospheric pressure. The plasma is maintained by an electric discharge between the spray capillary and a counter electrode. The ions are typically extracted into the mass analyzer by use of a set of differentially pumped skimmer stages. A counterflow of dry and preheated N2 gas may be used to improve removal of solvent. The gas-phase ionization in APCI can be more effective than ESI for analyzing less-polar entity.

“Multiple reaction mode” or “MRM” is a detection mode for a MS instrument in which a precursor ion (also referred to as parent ion) and one or more fragment ions are selectively detected and/or quantified.

Since a mass spectrometer separates and detects ions of slightly different masses, it easily distinguishes different isotopes of a given element. Mass spectrometry is thus, an important method for the accurate mass determination and characterization of analytes, including but not limited to low-molecular weight analytes, peptides, polypeptides or proteins. Its applications include the identification of proteins and their post-translational modifications, the elucidation of protein complexes, their subunits and functional interactions, as well as the global measurement of proteins in proteomics. De novo sequencing of peptides or proteins by mass spectrometry can typically be performed without prior knowledge of the amino acid sequence.

Mass spectrometric determination may be combined with additional analytical methods including chromatographic methods such as gas chromatography (GC), liquid chromatography (LC), particularly HPLC, and/or ion mobility-based separation techniques.

In the context of the present disclosure, the sample may be a sample derived from an “individual” or “subject”. Typically, the subject is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In preferred embodiments, the sample is derived from a human.

The term “chromatography” refers to a process in which a chemical mixture carried by a liquid or gas is separated into components as a result of differential distribution of the chemical entities as they flow around or over a stationary liquid or solid phase.

The term “liquid chromatography” or “LC” refers to a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). Methods in which the stationary phase is more polar than the mobile phase (e.g., toluene as the mobile phase, silica as the stationary phase) are termed normal phase liquid chromatography (NPLC) and methods in which the stationary phase is less polar than the mobile phase (e.g., water-methanol mixture as the mobile phase and C18 (octadecylsilyl) as the stationary phase) are termed reversed phase liquid chromatography (RPLC).

“High performance liquid chromatography” or “HPLC” refers to a method of liquid chromatography in which the degree of separation is increased by forcing the mobile phase under pressure through a stationary phase, typically a densely packed column. Typically, the column is packed with a stationary phase composed of irregularly or spherically shaped particles, a porous monolithic layer, or a porous membrane HPLC is historically divided into two different sub-classes based on the polarities of the mobile and stationary phases, namely NP-HPLC and RP-HPLC.

Micro LC refers to a HPLC method using a column having a narrow inner column diameter, typically below 1 mm, e.g. about 0.5 mm. “Ultra high performance liquid chromatography” or “UHPLC” refers to a HPLC method using a high pressure of e.g. 120 MPa (17,405 lbf/in²), or about 1200 atmospheres.

Rapid LC refers to an LC method using a column having an inner diameter as mentioned above, with a short length<2 cm, e.g. 1 cm, applying a flow rate as mentioned above and with a pressure as mentioned above (Micro LC, UHPLC). The short. Rapid LC protocol includes a trapping/wash/elution step using a single analytical column and realizes LC in a very short time<1 min.

Further well-known LC modi include Hydrophilic interaction chromatography (HIC), size-exclusion LC, ion exchange LC, and affinity LC.

LC separation may be single-channel LC or multi-channel LC comprising a plurality of LC channels arranged in parallel. In LC, analytes may be separated according to their polarity or log P value, size or affinity, as generally known to the skilled person.

As used herein “detecting” or“to detect” one or more analytes, such as, e.g., one or more steroids, in a sample at least means to determine whether the one or more analytes are present or absent in the sample. Detecting an analyte may or may not include quantifying said analyte, i.e. determining the absolute or relative amount of the analyte.

As used herein “quantifying” or “to quantify” one or more analytes, such as, e.g., one or more steroids, in a sample means to determine the presence and amount of said one or more analytes in the sample. The amount may be an absolute or relative amount of the analyte in the sample. The absolute amount can be any quantitative measure such as, for example, a concentration or mass. The relative amount may be any relative quantitative measure. For instance, the amount of the analyte may be detected relative to the amount of another sample ingredient, an internal standard added to the sample or a reference sample comprising the same one or more analyte.

“Final concentration” as used herein in the context of adding an agent and/or composition to a sample refers to the concentration of the agent and/or composition in the mixture obtained by adding said agent and/or composition to the sample.

The term “solvent” includes any solvent or mixture of solvent that keeps the analytes of interest (e.g. the one or more steroids) in solution. Exemplary but non-limiting examples for solvents or components of mixtures of solvents are water, alcohols (e.g. methanol or ethanol) and acetonitrile.

“Steroids” are a group of molecules known in the art. A steroid is an organic compound with a core structure of typically four rings, also referred to as steroid rings A, B, C and D. The core ring structure of steroids is typically composed of seventeen carbon atoms, which are bonded in four fused rings: three six C-atom cyclohexane rings (rings A, B and C) and one live C-atom cyclopentane ring (ring D). Steroids vary by the functional groups attached to its four rings and by the oxidation state of the rings. Some steroids also comprise changes to the ring structure in that one of the four rings is open. For instance, an open ring B is found in secosteroids one of which is vitamin D3.

Major classes of steroids known in the art are corticosteroids (e.g. glucocorticoids or mineralcorticoids), sex steroids (e.g. progestogens, androgens or estrogens), neurosteroids and secosteroids.

Classical androgens, as referred to herein, include Testosterone (T), Androstenedione (A4), Dehydroepiandrosterone (DHEA), Dehydroepiandrosterone sulfate (DHEAS) and Epitestosterone (ET).

The term “11-oxygenated steroids”, as used herein, includes all steroids that comprise a hydroxyl or a keton group at position 11 of the steroid core ring system.

The term “11-oxygnated C19 steroids”, as used in the present disclosure, relates to all steroids that have 19 carbon atoms in their chemical structure and that comprise a hydroxyl or a keton group at carbon position 11 of the steroid core ring system. Exemplary but non-limiting 11-oxygenated C19 steroids known in the art are 11β-Hydroxyandrostenedione (11-OHA4), 11-Ketotestosterone (11KT), 11-Ketoandrostenedione (11KA4), and 11β-Hydroxytestosterone (11OHT), 11-ketodihydrotestosterone (11KDHT) and 11β-hydroxydihydrotestosterone (11OHDHT). The structure of exemplary C19 steroids are depicted in FIG. 1(b).

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include the respective terms also in plural, unless the content clearly dictates otherwise.

Further, as used in the following, the terms “particularly”, “more particularly”, “specifically”, “more specifically” or similar terms are used in conjunction with features of particular or alternative embodiment(s), without restricting alternative possibilities. The disclosed method/system may, as the skilled person will recognize, be performed by using alternative features. Similarly, features introduced by “in an embodiment of the disclosed method/system”, “in embodiments” or similar expressions are intended to be additional and/or alternative features, without any restriction regarding alternative embodiments, without any restrictions regarding the scope of the disclosed method/system and without any restriction regarding the possibility of combining the features introduced in such way with other optional or non-optional features of the disclosed method/system.

Percentages, concentrations, amounts, and other numerical data may be expressed or presented herein in a “range” format. In the context of the present disclosure is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “4% to 20%” should be interpreted to include not only the explicitly recited values of 4% to 20%, but to also include individual values and sub-ranges within the indicated range. Thus, included in this numerical range are individual values such as 4, 5, 6, 7, 8, 9, 10, . . . 18, 19, 20% and sub-ranges such as from 4-10%, 5-15%, 10-20%, etc. This same principle applies to ranges reciting minimal or maximal values. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The following examples and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 (a) shows a scheme of the enzymatic biosynthesis of C19 steroids in the adrenocortical zona reticularis based on Pretorius et al. (2017, Mol Cell Endocrinol, 441: p. 76-85) and Rege et al. (2013. J Clin Endocrinol Metab, 98(3): p. 1182-8. C19 steroids are derived from cholesterol (27-carbon molecule) which is converted to DHEA (19-carbon molecule), following oxidative P450-catalyzed cleavage reactions, 11-oxygenated steroids are highlighted by a grey box. These compounds only differ in the C11- and C17-oxidation levels as shown in the right figure. Note that 11OHA4 and 11KT share the same molecular mass.

FIG. 1(b) depicts the chemical structure of four 11-oxygenated steroids. The carbon positions are numbered, 11 oxygenated steroids are characterized by the hydroxyl or keton group as substituent of C11 (here referred to as R1).

FIG. 2 shows a fragment ion scan (also referred to as product ion scan herein) for 11KA4 recorded by manual tuning in the mass range of m/z 50-350. The parent ion (M+H⁻−m/z 301.2) was fragmented using 36 CE Volts which results in the formation of prominent fragment ions (also referred to herein as product ions). Single and double water loss of the parent ion gives rise to m/z 283 and m/z 265, respectively. Cleavage of an ethoxy-portion (C2H4O—) yields the m/z 257 fragment. Separation of ring A from the steroid backbone yields m/z 121 which is stabilized by its tropylium-like cation structure.

FIG. 3 shows product ion scans of 11OHA4, 11KT and 11OHT (from top to bottom) recorded by manual tuning along the mass range m/z 50-350 at 36 CE volts.

FIG. 4 shows a calculated and experimentally observed LC retention pattern of steroid hormones. Based on measured chromatograms ChromSword® calculated a theoretical 1.2 min linear gradient and predicted the elution order of selected steroid hormones (chromatogram a). To a great extent the calculated elution order was confirmed by experimental findings (chromatogram b). The latter was recorded in two periods, indicated by the dashed lines. In the second period, negative ionization mode was used to measure DHEAS, leaving all other analytes to be measured in positive ionization mode. ISTDs of 11KT and 11OHA4 were added as bottom chromatogram in the first period.

FIG. 5: Processing of DoE results including blocked data. For the assessment of wash conditions sample preparation was conducted in blocks (whole plots). The summary of effects is shown in plot (a) and the respective actual by predicted plot below (plot b). Each whole plot represents a different wash solution (1-9). Each whole plot is shown in a different symbol.

FIG. 6: Comparison of predicted (left) and observed (right) data for the selection of wash parameters. (a) A section of the prediction profiler is shown on the left focusing on the influence of the organic content (% org) and pH of the wash solution as well as the bead type (BD) on the calculated concentration of 11OHT, T and DHEAS. Maximal analyte concentration is achieved by the addition of 5% organic solvent and under acidic conditions. (b) The measured chromatograms of 11OHT confirm the advantages of low wash pH. Abbreviations: pH3=acidic, pH7=neutral. pH10=basic.

FIG. 7: Comparison of predicted and observed data for the evaluation of elution parameters. (a) The impact of bead elution related parameters, i.e. content of organic solvent (% org), elution reagent (EL), bead type (BD) and addition of formic acid (FA), on the calculated concentration is shown by the prediction profiler. (b) The effect summary suggests that the effect % org is most significant throughout the data set. This is confirmed by the graph (c), even though profiled curves (dashed line) deviate somewhat from the observed values (black dots). Abbreviations. M=MeOH, A=ACN.

FIG. 8: Comparison of bead A and B characteristics. FIG. 8.1: Absolute intensity of DHEAS signal highly depends on the selected bead type. Magnetic bead A nearly causes detector saturation of DHEAS signal (5*10e7) whereas it drops down to a low but still detectable signal when magnetic bead B is used. FIG. 8.2: For the transition m/z 292.2—>256.2 an unspecific peak was recorded at retention time (RT)=0.85 min which might impair the detection of DHEA-13C3 (left peak) or ET-13C3 (right peak) if the column performance decreases. As indicated by the list of chromatograms, less interference was eluted from bead B than bead A, independently from the amount of organic solvent.

FIG. 9: Comparison or direct injection (DI) with two evaporation (Evap) scenarios. Left chart: The D1 workflow was conducted without evaporation and reflects the standard enrichment workflow. The evaporation experiments only differ in their EL and SN volumes where Evap2 has utilized 60 μL more than Evap1 Consequently, the relative transferred SN fractions as well as the calculated concentrations were greater for Evap2 Bars are flagged by numbers which indicate the specific enrichment factor (SEF) between evaporation and direct injection. Evaporation also did not lead to baseline signal increase as shown by the TIC chromatograms on the right.

FIG. 10: Recovery or androgenic steroids using the developed sample preparation method. Most steroids were recovered by 70%-75% 11OHT was recovered not as efficient as the other 11-oxy steroids (ca. 60%). A recovery gap was observed (ca, factor 700) between DHEAS and 11-oxy steroids. Yet, this is not problematic in view of the high DHEAS concentrations in serum samples.

FIG. 11: Comparison of 1 periodic with 2 periodic MRM settings.

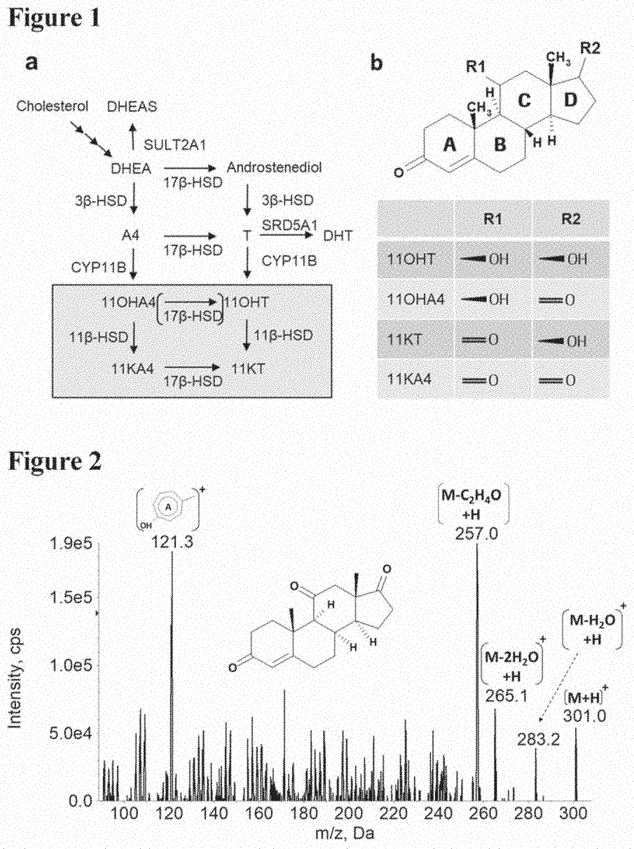

Insertion of an additional period increased the dwell time for 11KT from 2 ms to 15 ms. The increased dwell times reduce the noise level while at the same time increasing the signal height. Both peaks are described by the same number of data points, though.

### EXAMPLES

### Materials and Methods Used in the Examples

Reference Compounds and Internal Standards

Steroid reference compounds for generating spiked samples and for MS tuning as well as internal standards (ISTDs) were purchased from various vendors or synthesized in house; see tables 1 and 2, respectively. The chemicals were obtained with at least 97% purity. Isotopic purity of ¹³C₃-labeled compounds was at 100%, whereas deuterated ISTDs were at least 99.6% isotopically pure. For the 11-oxygenated C19 steroids 11 OHA4, 11KT, 11KA4 and 11OHT deuterated 11OHA4 and 11KT were chosen as ISTDs.

Primary stock solutions of 11OHA4, 11KT, 11KA4, 11OHT and the corresponding deuterated ISTDs, 11OHA4-d4 and 11KT-d3, were prepared in ethanol at 1 mg/mL and stored at −20° C. until used. All other reference analytes and ISTD were provided as methanolic 1 mg/mL solutions. Further dilution into working standards (10 μg/mL and 100 ng/mL) was performed using methanol. 1 μg/mL mixed solutions of either analytes or ISTDs were prepared in MeOH, too. These solutions were used to spike serum samples to generate samples to work with including calibrators or quality controls (QCs).

For quantification experiments, the ISTD was added to the sample in a defined amount prior to the magnetic bead workflow to ensure that the ISTD equilibrates with matrix and that analyte and ISTD were subjected to the same processing steps. In experiments that were conducted with samples having known concentrations of analytes and aimed to evaluate analyte recovery, the ISTDs were added after the magnetic bead workflow and, if used, the evaporation step.

TABLE 1
Reference compounds used in the Examples

| | |
|---|---|
| 11β-Hydroxyandrostenedione (11OHA4) | Sigma Aldrich (Darmstadt) |
| 11-Ketotestosterone (11KT) | Sigma Aldrich (Darmstadt) |
| 11-Ketoandrostenedione (11KA4) | Sigma Aldrich (Darmstadt) |
| 11β-Hydroxytestosterone (11OHT) | AHH Chemicals (ChangZhou, |
| | China) |
| Testosterone (T) | Sigma Aldrich (Darmstadt) |
| Androstenedione (A4) | TCI chemicals (Tokyo, Japan) |
| Dehydroepiandrosterone (DHEA) | TCI chemicals (Tokyo, Japan) |
| Dehydroepiandrosterone sufate | Avanti Polar (Alabaster, |
| sodium (DHEAS) | Alabama) |
| Epitestosterone (ET) | Sigma Aldrich (Darmstadt) |

TABLE 2
Internal standards used in the Examples

| | |
|---|---|
| 11OHA4-d₄ (9,11,12,12-d₄) | Cambridge Isotope Laboratories (Tewksbury, |
| | Massachusetts) |
| 11KT-d₃ (16,16,17-d₃) | Cayman chemical (Ann Arbor, Michigan) |
| T-13C₃ (2,3,4-¹³C₃) | Sigma Aldrich (Darmstadt) |
| A4-¹³C₃ (2,3,4-¹³C₃) | produced inhouse |
| DHEA-¹³C₃ (2,3,4-¹³C₃) | produced inhouse |
| DHEAS-¹³C₃ (2,3,4-¹³C₃) | produced inhouse |
| ET-¹³C₃ (2,3,4-¹³C₃) | produced inhouse |

Solvents

LCMS grade methanol (MeOH) and acetonitrile (ACN) were purchased from Biosolve (Valkenswaard, Netherlands). Ethanol (EtOH) in LCMS grade, DMSO (99%) and a 25% NH₃ solution were purchased from Merck (Darmstadt, Germany). Formic acid (FA, 99%) was purchased from VWR (Darmstadt, Germany) and deionized water was produced by a benchtop water purification system (Milli-Q® Advantage A 10) according to the manufacturer's instructions.

Magnetic Beads

Two magnetic bead types (type A and B) were used for sample preparation processes. Both bead types exert their chemical adsorption function by a polymer matrix layer around the magnetic core, which has a type dependent chemical modification (—OH for bead type A and two positively charged nitrogens for bead B). 50 mg/mL bead suspensions were stored and applied at pH 7.4 in potassium phosphate buffer.

Both beads type A and B comprised a porous polymer matrix embedding the magnetic core. The porous polymer matrix of beads of type A comprised pores having a pore size smaller than 100 nm as determined according to ISO15901. The porous polymer matrix of beads of type B comprised pores having a pore size from 0.5 nm to 50 nm as determined according to ISO15901.

The porous polymer matrix of both bead types comprised a crosslinked polymer comprises a co-polymer obtained by a polymerization of vinylbenzylchloride and divinylbenzene. Further, both bead types were hypercrosslinked.

Type A beads were functionalized with —OH on their surface. Type b beads not. Instead, for these beads type B hypercrosslinking was performed such that two positively charged nitrogens are present in the hypercrosslink bond.

Beads of type A were produced by the methods as described in WO2018189286A 1. Beads of type B were produced by the methods as described in WO2019141779A 1.

Human Serum Samples

Two types of pooled human serum were used for method development, stripped and native serum. Stripped serum has undergone several purification steps including filtering through active carbon to remove endogenous components such as steroids. Native serum used for assay development was based on pooled native individual serum. Each serum was drawn from approximately 50 healthy patients, stored in 20 or 50 mL falcons at −80° C. and thawed when needed.

Handling During Sample Preparation

Sample preparation steps prior to LC-MS/MS analysis were performed manually or using fully automated pipetting using a sample preparation breadboard (SPBB). The SPBB hardware is a Hamilton pipetting platform (Nevada, USA).

Concentration of Analytes by Evaporation

In the experiments using evaporation the eluate from the magnetic bead workflow was evaporated for 1 h at 45° C. down to a pressure of 20 Torr using a SpeedVac Concentrator SPD 2010 from Thermo Fisher Scientific (Waltham, USA). After evaporation to dryness samples were resolubilized in a respective volume of organic solvent and shaked for 10 min on a Roller 10 digital from IKA (Staufen, Germany).

Mass Spectrometry

Mass spectrometric detection was carried out using a Triple Quad 6500+ LC-MS/MS system from AB Sciex (Darmstadt, Germany) The Analyst® software (version 1.6.3) from AB Sciex was used to control the instrument and to visualize data.

High-Performance Liquid Chromatography (HPLC)

High-performance liquid chromatography (HPLC) was performed on an Agilent 1200 Infinity 11 LC System (Waldbronn, Germany) comprising a multisampler, a multicolumn thermostat and a binary high pressure gradient pump. The autosampler maintained 8° C. in its sample compartment. The instrument was controlled via the Analyst device driver from AB Sciex. Chromatographic separation was performed using a C18 HPLC column (2.1 mm i.d.×50 mm) packed with SunShell 2.6 μm fused core particles from ChromaNik (Osaka, Japan). 0.1% formic acid (FA) (A) and MeOH with 0.1% FA (B) were used as mobile phases. The column oven temperature was set to 50° C. The LC gradient was established by mixing defined amounts of (A) and (B).

Data Processing, Evaluation and Statistics

Peak picking, integration and calibration was performed using MultiQuant™ (AB Sciex. Darmstadt, Germany). The integration parameters were adjusted individually due to differences in the baseline height and amount of interfering peaks. In general, the integration retention time (RT) half window was set to 3 seconds for those transitions with neighboring signals and 5-10 seconds if there were none. The minimum peak height was set double the height as the baseline signal. No smooth filter was applied to the data. Linear calibration curses were designed based on the area ratio of analyte and ISTD without curve weighting. Subsequent calculation of absolute concentrations of unknown samples was conducted automatically.

Statistical analysis and experiment planning was performed in JMP®, version 12.1.0 (Cary, North Carolina). For development of the experimental setups testing different parameters the custom Design of experiment (DoE) platform of JMP® was employed. Data were collected according to run conditions proposed in the DoE matrix, processed in MultiQuant™ and fed into JMP®'s DoE data tables. The model calculation with JMP®, finally, used the experimental data to predict factor interactions.

All types of experiments, including batch files for LC-MS analysis or run lists for automated sample preparation, were completely randomized to prevent generation of biased data.

Absolute Quantification

Absolute analyte concentrations were calculated with the aid of internal standards (ISTDs) that were added to the sample before sample preparation (i.e. before the SPE by magnetic bead workflow) in a defined amount.

Before starting determination of unknown analyte concentrations Cx, a calibration using samples with known analyte concentrations was conducted to calculate the response factor R (Equation 1). This factor compares the ratio of peak areas A to the ratio of concentrations C (1.1). Through variation of the analyte concentration and analyte area (Cx and Ax) in the samples measured for calibration, a calibration plot (Ax/AISTD vs Cx/CISTD) could be calculated R was determined by the slope of the calibration curve. Given that R and CISTD were known quantities, the absolute concentration of the analyte Cx could be calculated by the area ratio of the analyte to internal standard.

$$R = \frac{{A}_{x} / {A}_{ISTD}}{{C}_{x} / {C}_{ISTD}} \qquad ( 1.1 )$$

$${C}_{X} = \frac{{A}_{x} / {A}_{ISTD}}{R} \cdot {C}_{ISTD} \qquad ( 1.2 )$$

$$Calculation of absolute concentrations {C}_{x} using an \\ internal standard . \qquad Equation 1$$

The response factor R compares the ratio of peak areas A to the ratio of concentrations C and is determined during calibration (1.1). Whereas R and C_{ISTD} are known factors the analyte concentration Cₓ is unknown, but can be calculated by the ratio of Aₓ to A_{ISTD} (1.2).

The ISTD which eluted closest to the retention time of an analyte was chosen as the quantifying reference standard the respective analyte in the present examples. Given the observed retention times using the LC conditions elaborated in Example 2, 11-KT-d3 was used as ISTD for the quantification of 11-KT and 11-KA4 and 11OHA4-d4 was used as ISTD for 11OHA4 and 11OHT herein. However, also other standards could be used, as indicated herein above.

The ISTD concentration was set to 30% of the highest calibrator concentration, i.e. 600 ng/mL or 60 ng/mL in case of DHEAS and 6 ng/mL in case of other steroid metabolites.

Determination of Sensitivity

Two distinct criteria were used for determining sensitivity, the signal-to-noise (S/N) ratio and the coefficient of variance (CV) of area ratios. The limit of quantification (LOQ) was defined as the lowest concentration at which a S/N ratio greater 5 is measured and the area ratio of results deviates with less than 20% CV.

Determination of Precision

Precision was defined as the deviation of replicate samples from each other based on statistical errors, i.e. the deviation of single results around their arithmetic mean. It was determined by calculation of the coefficient of variance (CV).

Determination of Accuracy

Accuracy was defined as the deviation of the arithmetic mean, i.e. the average of calculated concentrations, from the true value or the true concentration caused by systematic errors. It was expressed by the percent error (% error) which is the relative deviation of the observed or calculated concentration Cobserved from the true concentration Ctrue (Equation 2).

$$\% error = \frac{( {C}_{observed} - {C}_{true} )}{{C}_{true}} \cdot 100 \\ calculation of the percent error ( \% error ) . \qquad Equation 2$$

Determination of Analyte Recovery

Recovery was defined as the ratio of absolute amounts for each of the steroids after and before sample preparation (Equation 3), i.e. the steroid amount in processed serum/sample (PS) and native serum/sample (serum).

$$Recovery = \frac{{C}_{PS} \times {V}_{PS}}{{C}_{serum} \times {V}_{serum}} \cdot 100 \qquad ( 4.1 )$$

$$EF = \frac{{C}_{PS}}{{C}_{Serum}} \qquad ( 4.2 )$$

$$Recovery and enrichment factor ( RF ) calculation . \qquad Equation 3$$

### Example 1: MS Tuning and Selection of Ions for Analyte Detection

The analytes listed in Table 1, above, were tuned in the mass spectrometer to select a set of mass transitions for each analyte that are best suitable for quantification and/or detection. The best transitions for quantification and detection of the different analytes were selected with respect to their relative abundance and their fragmentation characteristics.

For tuning, the steroid hormones and ISTDs listed in Tables 1 and 2 were dissolved in MeOH to a final concentration of 100 ng/mL. The solutions were consecutively infused to the mass spectrometer using a syringe pump at a constant flow rate of 10 μL/min. The mass spectrometer's ion source was operated in positive ESI mode with the only exception that DHEAS was measured in a negative ion mode. Curtain gas was kept at 20 psi whereas nebulizer and turbo gas were set to 10 psi. A voltage of 4500 V was applied to the ESI needle while the gas temperature was held at 100° C. Subsequently, an automatic tuning procedure was started which includes an m/z scan in order to select the most abundant product ions for a preselected parent ion m/z. All automatic tuning processes used the [M+H]⁻ adduct as parent ion. Using the [M+H]⁻ parent ion turned out to be more sensitive than using the [M−H2O+H]⁻ species as parent ion. Product ions were excluded within a range of +/− 20 m/z of the monoisotopic parent ion mass and below m/z 80 in order to prevent automatic selection of [M−H2O+H]⁻ ions on the one hand and unspecific low mass fragment ions on the other hand. For each mass transition, the particular voltages for subsequent stages of the ionization and detection process, which are droplet declustering potential (DP), quadrupole entry potential (EP), ion collision energy (CE) and quadrupole exit potential (CEP) were obtained by the Analyst software (version 1.6.3) from AB Sciex. These voltage values are instrument specific and can be adapted with routine tuning when another mass spectrometer is used.

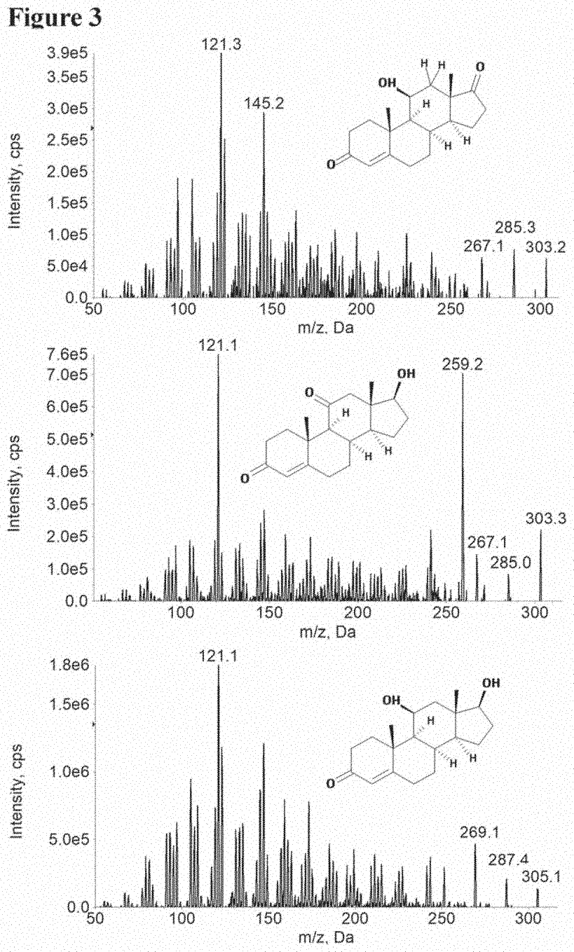

Tuning the mass spectrometer's MRM settings yielded a number of MRM transitions per analyte. The tuning result for the 11-oxygenate C19 steroids are depicted in FIGS. 2 and 3. For each analyte and ISTD the two most abundant transitions (with some exceptions) were selected with respect to their signal to noise ratio. Due to the similarity of 11-oxygenated steroids, those two transitions resulted in characteristic product ions, one at m/z 121 and the other above m/z 250. The transition which was more abundant, i.e. yielded a greater signal while it was tuned, was used for quantification (quantifier). The second transition was used as qualifier to aid proper identification of the analyte. This approach further improves the selectivity of the method by avoiding false positive results.

Selectivity is also provided by the fact that at least one transition, either quantifier or qualifier, yields a product ion above m/z 250. Unlike the m/z 121 ion, which is common to many steroid analytes, this product ion is structurally closer to the original analyte and helps to distinguish between several 11-oxygenated steroids.

The mass transitions selected in the tuning procedure for the further examples are summarized in Table 3 below. The DP, EP, CE and CXP values were specific for the MS instrument used. A skilled person is capable of defining similar settings on other MS devices.

TABLE 3
Major MRM transitions and corresponding detector
voltages used for MS data acquisition. The first
transition of each compound is best performing
(most abundant transition) and set as quantifier
whereas the other serves as qualifier (second
most abundant). All transitions are recorded in
positive mode, with the exception of DHEAS (*).

| | Q1 | Q3 | | | | |
|---|---|---|---|---|---|---|
| | mass | mass | | | | |
| Analyte/ISTD | (m/z) | (m/z) | DP | EP | CE | CXP |
| 11OHA4 | 303.1 | 267.2 | 80 | 10 | 25 | 16 |
| | 303.1 | 121.1 | 80 | 10 | 35 | 14 |
| 11KT | 303.1 | 121.1 | 100 | 10 | 33 | 15 |
| | 303.1 | 259.2 | 100 | 10 | 31 | 15 |
| 11KA4 | 301.05 | 257.2 | 80 | 10 | 31 | 14 |
| | 301.05 | 121.2 | 80 | 10 | 33 | 14 |
| 11OHT | 305.2 | 269.2 | 100 | 8 | 23 | 10 |
| | 305.2 | 121.1 | 100 | 8 | 29 | 10 |
| T | 289.2 | 97.1 | 90 | 10 | 27 | 16 |
| | 289.2 | 109.1 | 90 | 10 | 31 | 8 |
| A4 | 287.2 | 97.1 | 96 | 10 | 29 | 12 |
| | 287.2 | 109.1 | 96 | 10 | 31 | 8 |
| DHEA | 289.2 | 213.1 | 60 | 10 | 25 | 14 |
| | 289.2 | 91 | 60 | 10 | 63 | 16 |
| DHEAS* | 367.2 | 80.1 | −95 | −10 | −130 | −11 |
| | 367.2 | 97.1 | −95 | −10 | −40 | −7 |
| ET | 289.2 | 109 | 73 | 10 | 33 | 12 |
| | 289.2 | 97 | 73 | 10 | 29 | 14 |
| 11OHA4-d₄ | 307.1 | 121.1 | 80 | 10 | 35 | 15 |
| | 307.1 | 148.2 | 80 | 10 | 33 | 15 |
| 11KT-d₃ | 306.1 | 262.2 | 100 | 10 | 33 | 12 |
| | 306.1 | 121.1 | 100 | 10 | 31 | 12 |
| T-¹³C₃ | 292.2 | 100 | 45 | 10 | 29 | 12 |
| | 292.2 | 112.1 | 45 | 6 | 33 | 12 |
| A4-¹³C₃ | 290.1 | 100 | 96 | 10 | 29 | 12 |
| | 290.1 | 112.1 | 96 | 10 | 31 | 8 |
| DHEA-¹³C₃ | 292.2 | 256.2 | 60 | 10 | 17 | 16 |
| | 292.2 | 216.4 | 60 | 10 | 25 | 14 |
| DHEAS-¹³C₃* | 370.2 | 97 | −60 | −10 | −38 | −5 |
| | 370.2 | 80 | −60 | −10 | −130 | −11 |
| ET-¹³C₃ | 292.2 | 100.1 | 60 | 10 | 30 | 12 |
| | 292.2 | 112 | 60 | 10 | 34 | 14 |

### Example 2: LC Settings

To obtain a robust and sensitive detection of 11-oxygenated C19 steroids and classical steroids, in silico predictions and experiments defining LC settings were performed. The aim was to separate the steroids to be analyzed in a short time (<2 min) and in a manner that no isobaric steroids or steroids that have isobaric fragmentation ions co-elute. The short time helps to increases sample throughput.

To determine LC settings with the desired performance, two exemplary chromatograms were recorded under predefined conditions (gradients A and B) and the data was subjected to an in silico LC setting prediction using the software ChromSword® (S. Galushko & Merck. Darmstadt) and further manually optimized.

The two input chromatograms for the in silico prediction were obtained as follows. A steroid mix including all steroid hormones of the analyte panel (see Table 1) was injected. Baseline separation of steroid hormones was achieved using a linear 3 min and 9 min LC gradient (gradient A and B in Table 4 below), from 2% B to 98% B at 1 mL/min followed by a 1.5 min isocratic wash period and subsequent 0.6 min for column equilibration at 2% B. Retention times, peak width and area were extracted from the chromatograms using MultiQuant™ and entered into the ChromSword® software. Dwell time and zero time were determined as 0.09 min and 0.105 min for the given set-up and were also entered into the ChromSword® software. Dwell time describes the time between the point of mixing in the pump and the top of the column, i.e. the time the gradient needs to become “active” Zero time was considered as the time a non-retained substance requires from injection until detection.

Using the recorded chromatograms with gradients A and B as input for the in silico prediction. LC conditions that achieve the separation in a time range that is as short as possible were determined using the ChromSword® software and additional manual input. The predicted optimized LC settings (gradient C) are depicted in Table 4 below. The predicted gradient C incorporates a 1.2 min gradient which starts at 50% B and linearly rises up to 75% B. This predicted time frame is sufficient for baseline separation of 11-oxysteroids and would theoretically meet the requirement of max. 2 min at a flow rate of 1 mL/min.

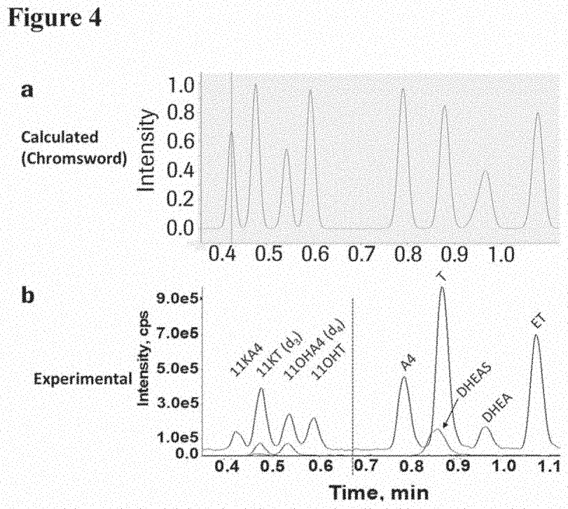

To verify that the newly established gradient C performs as predicted, the gradient was tested experimentally using the same steroid mix solution as for gradient A and B. A comparison of the predicted elution pattern and the experimental chromatogram is depicted in FIG. 4. FIG. 4 confirms that there is a good correlation between the calculated (top) and experimental (bottom) chromatogram. This is in particular the case for the 11-oxygenated C19 steroids, which are detected in the first half of the gradient. For all 11-oxygenated C19 steroids, even for the isobars 11KT and 11OHA4, good baseline separation was achieved.

In the second half of the LC gradient the classic androgens were recorded. The experimental chromatogram corresponds to the calculated retention pattern to a great extent. Only DHEAS, which is measured in negative mode co-elutes with T instead of DHEA. This co-elution does not affect the analysis, because DHEAS has unique ionization characteristics.

TABLE 4
LC gradients applied for the separation of serum steroids
during method development: The chromatograms of gradient
A and B (3 min and 9 min) served as references to figure
out ideal separation conditions (gradient C) using the gradient
software Chromsword ®. Gradient C are the conditions
that were finally selected for the further Examples

| Gradient A | | Gradient B | | Gradient C | |
|---|---|---|---|---|---|
| Time [min] | B [%] | Time [min] | B [%] | Time [min] | B [%] |
| 0.0 | 2 | 0.0 | 2 | 0.0 | 50 |
| 3.0 | 98 | 9.0 | 98 | 1.2 | 75 |
| 4.5 | 98 | 10.5 | 98 | 1.4 | 98 |
| 4.6 | 2 | 10.6 | 2 | 3.0 | 98 |
| 5.0 | 2 | 11.0 | 2 | 3.2 | 50 |
| | | | | 3.8 | 50 |

Chromatograms of 11KT-d3 and 11OHA4-d4 were also measured and are also depicted in FIG. 4. The results confirm that the ISTDs show the same retention as their corresponding analytes. Having the same retention for the analyte and the ISTD increases robustness of the method.

In sum, a gradient was established that allows robust separation of 11-oxygenated C19 steroids and other steroids. In fact, even a separation of two groups, the first one comprising the 11-oxygenated C19 steroids and the second one comprising other measured steroids was achieved. These two well separated groups of analytes facilitate period-wise data acquisition of the mass spectrometer, with a first period measuring 11-oxygenated steroids and a second period measuring other steroids.

### Example 3: MS Settings for LC-MS/MS Experiments

The MRM settings were adapted to the retention pattern of the steroid hormones as shown in FIG. 4. By means of chromatography, 11-oxygenated steroids and classic androgens were clearly separated into two distinct blocks of analytes which enter the mass spectrometer at different time points. The sensitivity of quadrupole based MS detectors greatly depends on the time the mass spectrometer spends on detection of a particular m/z ion.

Facilitated by the elution pattern, the MS method was consequently split into two periods. The first 0.55-0.70 min of the chromatogram were reserved for the detection of 11-oxy steroids and respective ISTDs while the second period (3.10-3.25 min) was devoted for the other steroid hormones (see FIG. 4).

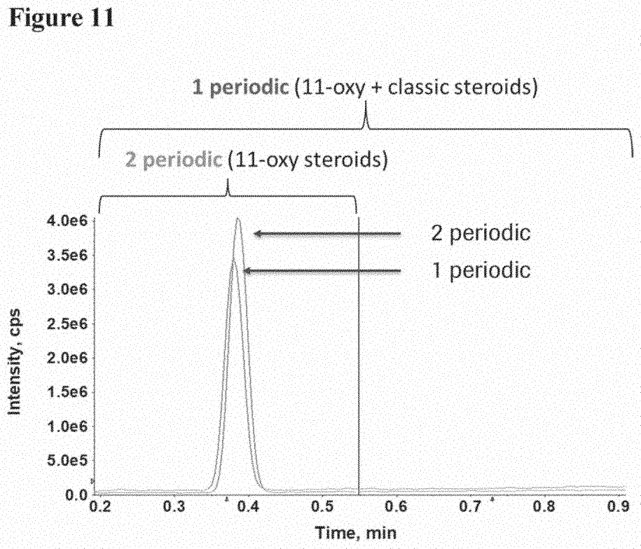

To illustrate the advantage of a two periodic measurement, a one and a two periodic measurement were compared. The results are depicted in FIG. 11 and illustrate the benefit in sensitivity achieved by the two periodic NIS. The mass spectrometer is less sensitive when using a single period instead of a two periodic MS method, which is shown by different peak intensities in the chromatogram. Correspondingly, the dwell time, which is the time for collection of a specific m/z ratio, increased from 2 ms to 15 ms by using the two periodic MS.

The cycle time was set to 170 ms for period one and 230 ms for period two in the further experiments.

### Example 4: Pretreatment with Organic Releasing Agent

The goal of this example was to evaluate the influence of a pretreatment step that aims to loosen interactions between steroids and binding proteins, such as the sex hormone binding globuline (SHBG) (Rwarola. J, steroid Biochem., 1983, 18, p. 5). Especially, the influence of a pretreatment with an organic solvent on the recovery of different steroids (namely 11-oxygenated steroids and classic androgens) using the mass spectrometry workflow method of the present invention was assessed. The aim of this examples was to evaluate the influence of a sample pretreatment influences the recovery of 11-oxygenated steroids and classical androgens in a mass spectrometry work-flow according to the invention. Interference with SHGB binding can be achieved by addition of organic solvents to the pretreatment solution, as previously reported (Gervasoni et al., Clin Biochem, 2016, 49 (13-14): p. 998-1003). The aim of the present example was to determine the influence of a sample pretreatment using organic solvents such as ACN on the recovery of different steroids. Pre-experiments (not shown) revealed that also other organic solvent than ACN can be used with a comparable performance (see also below). Thus, sample preparation using either 50 μl of 8 vol % ACN (as exemplary organic pretreatment) or no pretreatment (addition of water) was performed.

The sample preparation settings used are summarized in table 5 below:

TABLE 5
Sample prep workflow.

| | | Content of added | |
|---|---|---|---|
| SP step | Volume (μl) | volume | time (sec) |
| SA | 150 | Pooled native | 60 |
| | | serum | |
| PT | 50 | 8 vol % ACN | 50 |
| BD | 20 | Magnetic Bead | 250 |
| | | Type B | |
| Wash | 250 × 2 | purified water | 65 × 2 |
| EL | 60 | 80% MeOH or | 275 |
| | | 50% ACN | |
| SN | 30 | — | 40 |
| LC-Dil | 30 | 20% MeOH + | 40 |
| | | ISTD | |

SA: Sample, PT: Pretreatment, BD: Bead application, Wash: Wash step (repeated once), EL: Bead elution, SN: Supernatant transfer, LC-Dil: LC dilute (dilution of supernatant prior to LC injection), SPBB: Sample preparation breadboard.

The direct comparison of aqueous and organic pretreatment yielded the results shown in Table 6. For most androgens, quantification of the endogenous levels in the sample type and volume used was successfully accomplished. Only 11KA4, 11OHT and ET were below the limit of quantification (LOQ) in the mixed serum. Yet, by optimization of the sample preparation conditions. e.g. by using more sample volume and including a final evaporation step, also these analytes can likely be effectively detected and quantified.

As indicated by the p value, the organic pretreatment significantly increased the measured concentrations of T and DHEAS, respectively. The relative increase of recovered DHEAS concentration was about 15%. The recovered T increased even by about 23%. The DHEAS levels measured are generally low due to the use of beads type B for SPE enrichment of the steroids. Beads B do not recover DHEAS very efficiently. However, the lower recovery of DHEAS is not an issue because DHEAS is typically present in high concentrations and using an ISTD (addition prior to SPE rather than after in this example) one can still measure the correct DHEAS concentration.

Apart from T and DHEAS, the measured concentrations did not increase significantly with the application of 8 vol % ACN.

Additional experiments testing additional pretreatment conditions, such as 30 vol % MeOH or 20 vol % DMSO were also conducted. These experiments showed that the performance of these other organic pretreatments was similar to 8 vol % ACN.

In sum, the present example demonstrates that a pretreatment with an organic solvent improves recovery of some but not all steroids. Especially for the detection of 11-oxygenated steroids, pretreatments do not seem to have a major impact on analyte recovery. Thus, while a pretreatment with organic solutions is advantageous for certain steroids it is not mandatory.

TABLE 6
Experiment comparing the effect of aqueous (MilliQ water)
and organic pretreatment (addition of 8 vol % ACN) on
native serum. Sample prep was conducted using bead type
B without pH adjust. The mean and standard deviation
(SD) of androgen concentrations are given in ng/mL.

| Steroid analyte | MilliQ water | | 8% ACN | | |
|---|---|---|---|---|---|
| [ng/ml] | Mean | SD | Mean | SD | p value |
| 11KA4 | <LOQ | <LOQ | <LOQ | <LOQ | NS |
| 11KT | 0.32 | 0.01 | 0.32 | 0.02 | NS |
| 11OHA4 | 0.66 | 0.03 | 0.65 | 0.02 | NS |
| 11OHT | <LOQ | <LOQ | <LOQ | <LOQ | NS |
| T | 2.21 | 0.13 | 2.72 | 0.08 | <0.001 |
| A4 | 0.76 | 0.03 | 0.76 | 0.02 | NS |
| DHEA | 4.82 | 0.21 | 4.74 | 0.11 | NS |
| DHEAS | 0.39 | 0.02 | 0.45 | 0.04 | <0.001 |
| ET | <LOQ | <LOQ | <LOQ | <LOQ | NS |

### Example 5: Bead Type and Bead Wash

In this example, the use of two different types of beads and different wash conditions in the SPE step of the method of the invention was assessed in form of a Design of Experiment approach.

Specifically two types of magnetic beads (type A and 11; see Materials and methods, above) were tested and the wash buffers with three different pH values and three different concentrations of organic solvent were employed. The pH of the wash solution was adjusted with 40 mM formic acid for the acidic wash solutions and with 5 mM NH3 for basic wash solutions. As comparison, MilliQ water was used as wash solution. To establish 0, 5 and 10 vol % of organic content, methanol was added in respective amounts to the wash solution Table 7 lists all DoE factors and levels of this experiment. Besides the wash pH and % org, the bead type as well as the elution type was changed on two levels. As elution solution aqueous 50 vol % ACN and 80 vol % MeOH solutions were assessed.

TABLE 7
DoE factors and levels set for the bead wash experiment.

DoE factors

| | Wash pH | Wash % org | BD | EL |
|---|---|---|---|---|
| DoE | 2.6 (acidic) | 0 | Bead A | 50% ACN |
| levels | 7 (neutral) | 5 | Bead B | 80% MeOH |
| | 10.5 (basic) | 10 | | |

BD: Bead type, EL: Elution type, wash % org: content of MeOH in wash buffer (v/v).

The experimentally tested wash solutions besides MilliQ water are listed in Table 8 below.

TABLE 8
Composition of various wash solutions tested during sample prep
development. Wash solutions varied in their pH value and organic
content (% org). Low pH was established using 40 mM FA and high
pH using 5 mM NH3. Neutral pH was maintained using MilliQ water.
All organic solvents were prepared volumetric (v/v).

| Wash solution | pH | % org [%] |
|---|---|---|
| 1 | 7 | 5 |
| 2 | 7 | 0 |
| 3 | 2.6 | 5 |
| 4 | 10.5 | 5 |
| 5 | 2.6 | 0 |
| 6 | 2.6 | 10 |
| 7 | 7 | 10 |
| 8 | 10.5 | 0 |
| 9 | 10.5 | 10 |

Automated sample preparation was conducted according to the sample prep conditions as listed in Table 9, below.

TABLE 9
Sample prep workflow.

| | | Content of added | |
|---|---|---|---|
| SP step | Volume (μl) | volume | time (sec) |
| SA | 150 | Pooled native | 60 |
| | | serum (spiked | |
| | | with analyte) | |
| PT | 50 | 8 vol % ACN | 50 |
| BD | 20 | Magnetic Bead | 250 |
| | | type A or B | |
| Wash | 250 × 2 | MilliQ water or | 65 × 2 |
| | | wash solution 1 to | |
| | | 9 (see Table 8) | |
| EL | 60 | 80% MeOH or | 275 |
| | | 50% ACN | |
| SN | 30 | — | 40 |
| LC-Dil | 30 | 20% MeOH + | 40 |
| | | ISTD | |

SA: Sample, PT: Pretreatment. BD: Bead application, Wash: Wash step (repeated once), EL: Bead elution, SN: Supernatant transfer, LC-Dil: LC dilute (dilution of supernatant prior to LC injection), SPBB: Sample preparation breadboard.

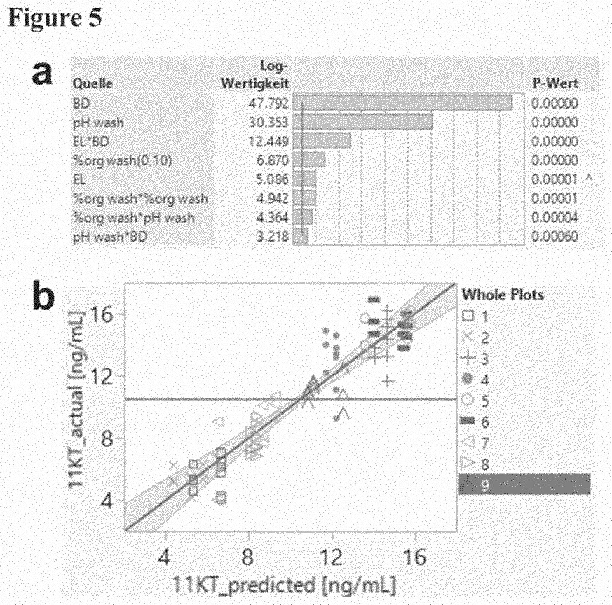

The DoE results are exemplary shown for 11 KT in FIG. 5 which comprises the effect summary (a) and the respective actual by predicted plot (b). Plot b shows that wash conditions can be distinguished from each other. The effect Summary in (a) illustrates that the bead type has the greatest influence on 11-KT recovery. pH adjust is the factor with second highest impact while the organic content (% org) and elution (EL) have less significant influence.

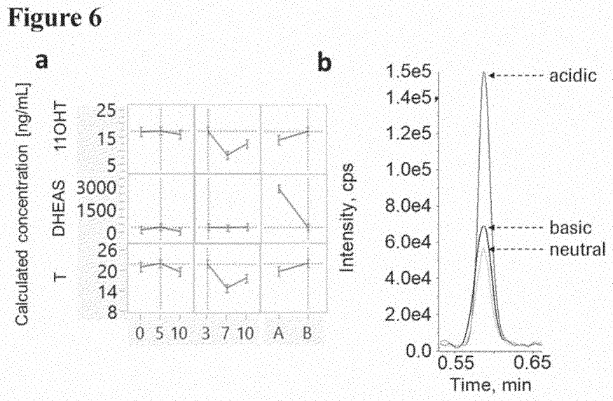

FIG. 6 shows a part of the predicted (a) and observed (b) data of this experiment. The prediction profiler plots in panel (a) show the calculated concentration (i.e. a measure for recovery) as response values in dependence of the factors percent organic % org), pH value (pH) and bead type (BD). The bead elution (EL) which is not illustrated in this figure was set to 80% MeOH. 11OHT and T are shown as representatives for 11-oxygenated C19 steroids and classic androgens, respectively. Similar data has also been obtained for the other representatives of these groups, i.e. 11β-Hydroxyandrostenedione (11-OHA4), 11-Ketotestosterone (11KT) and 11-Ketoandrostenedione(11KA4) for the 11-oxygenated steroids and Androstenedione (A4). Dehydroepiandrosterone (DHEA), and Epitestosterone (ET) for the classical androgens. The results for DHEAS, which is also a classical androgen are also shown in FIG. 6(a).

The data illustrates that over all steroids the maximal recovery was obtained with the addition of 5% organic solvent to the wash solution. Further, acidic conditions (pH 3) turned out to result in better recovery. Regarding the beads, type B shows the best recovery with the exception of DHEAS. For DHEAS, bead type A shows a significantly better recovery rate. FIG. 6(a) again shows that % org in the wash solution does not affect the calculated concentration as strong as the pH value or the bead type. These findings are in good agreement with the corresponding effect summary shown in FIG. 5(a).

Chromatograms of three different wash pH conditions are depicted in FIG. 11(b). The highest 11OHT peak is achieved using acidic wash conditions. The peaks recorded under neutral and basic conditions are only half the size. Background signal and peak shapes are quite comparable, though. The order of peak intensities obtained from the chromatograms confirms the order of calculated concentrations shown in FIG. 6(a).

### Example 6: Bead Type and Elution

In this example the influence of the bead type and the elution parameters in the magnetic bead based SPE on analyte recovery by the method of the invention were assessed.

Elution

A DoE-based experiment was conducted including those DoE factors listed in Table 10, below. The factor elution type (EL) was used to distinguish between the types of organic solvents. MeOH and ACN were chosen as corresponding factor levels. The second factor was the percent of organic solvent, abbreviated by % org which ranged from 10% b to 90%. It was supposed that this continuous factor has a quadratic effect on recovery, thus at least three organic concentrations, 10%, 50% and 90%, were necessary in the actual experimental data collected. Besides, the impact of 0.1% FA (v/v), which was added to the elution reagent, on recovery was tested. The bead type (BD) was the fourth factor with the values magnetic bead type A and magnetic bead type B as in the previous examples.

TABLE 10
DoE factors and levels set for the bead elution experiment.

DoE factors

| | % org | EL | FA | BD |
|---|---|---|---|---|
| DoE | 10 | MeOH | Yes | Bead A |
| levels | 50 | ACN | no | Bead B |
| | 90 | | | |

% org: content of organic in elution reagent, EL: Elution type, BD: Bead type, FA: addition of formic acid.

Combination of the various factor variables, eventually, yielded ten elution conditions to be tested, which are listed in Table 11. All eluents were prepared prior to the SPBB run.

TABLE 11
Composition of various eluents tested during sample prep development.
Organic solvents (elution types) were prepared in various concentrations
(% org) with or without the addition of 0.1% FA. Organic solvents
and FA were prepared volumetric (v/v).

| Eluent | Elution type | % org [%] | 0.1% FA |
|---|---|---|---|
| 1 | MeOH | 10 | No |
| 2 | MeOH | 90 | Yes |
| 3 | ACN | 10 | Yes |
| 4 | ACN | 90 | Yes |
| 5 | MeOH | 50 | No |
| 6 | ACN | 90 | No |
| 7 | MeOH | 90 | No |
| 8 | MeOH | 10 | Yes |
| 9 | ACN | 10 | No |
| 10 | ACN | 50 | No |

The measurements on which this DoE was based were conducted with the following workflow:

TABLE 12
Sample prep workflow.

| | | Content of added | |
|---|---|---|---|
| SP step | Volume (μl) | volume | time (sec) |
| SA | 150 | Pooled native | 60 |
| | | serum (spiked | |
| | | with analyte) | |
| PT | 50 | 8 vol % ACN | 50 |
| BD | 20 | Magnetic Bead | 250 |
| | | type A or B | |
| Wash | 250 × 2 | | 65 × 2 |
| EL | 60 | Eluent | 275 |
| | | 1, 2, 3, 4, 5, 6, 7, 8, 9 | |
| | | or 10 as listed in | |
| | | Table 11 | |
| SN | 30 | — | 40 |
| LC-Dil | 30 | 20% MeOH + | 40 |
| | | ISTD | |

SA: Sample, PT: Pretreatment, BD: Bead application, Wash: Wash step (repeated once), EL: Bead elution, SN: Supernatant transfer, LC-Dil: LC dilute (dilution of supernatant prior to LC injection), SPBB: Sample preparation breadboard.

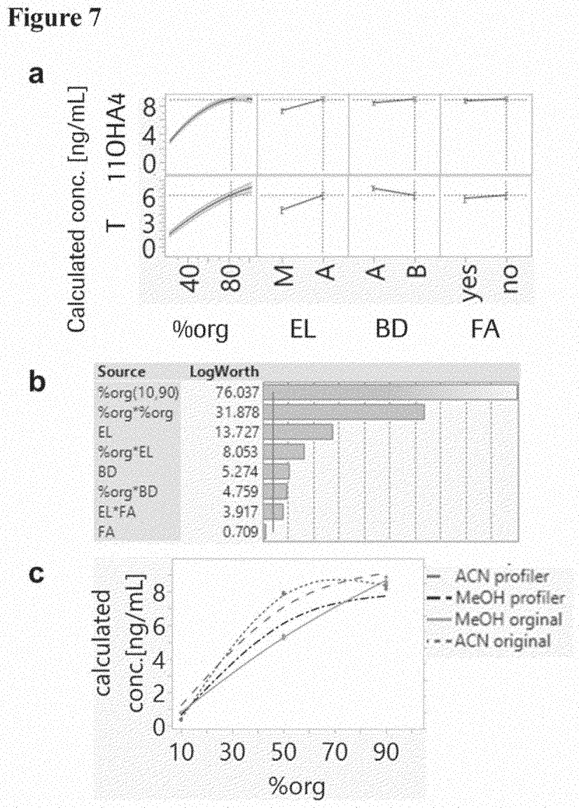

The results of the DoE are depicted in FIG. 7. FIG. 7(a) shows the prediction profiler which plots the calculated concentration as response value of the investigated factors, i.e. percent of organic content (% org), elution type (EL), bead type (BD) and addition of 0% formic acid (FA). The data is again exemplary depicted for one member of the 11-oxygenated (C19 steroids and the classical androgens, respectively. As shown in the effect summary of FIG. 7(b) % org has the highest impact on the response. This statement is confirmed by the proposed function in the % org column of the profiler in FIG. 7(a). It suggests that the measured concentration of T and 11OHA4 increases with rising organic solvent content. Moreover, % org is calculated to have a quadratic impact on the measured concentration which is recognizable both by the profiler's curve shape and by the second highest entry of the effect summary (org*org). Compared to % org, the elution type and bead type explain a lower proportion of data separation, in decreasing order, which can be inferred from lower LogWorth values (see FIG. 7(a)). Addition of formic acid to the eluate was calculated to have no significant effect on recovery. In order to obtain maximal concentration the JMP® software recommends based on the measured and selected factors to use as much ACN as possible, i.e. 90 vol %. This is preferably combined with bead B. FA addition to the elution solution is predicted to be not beneficial. ACN and MeOH have both a good elution performance. Yet, FIG. 7(a) and 7(c) show that ACN has a slightly stronger elution capacity as MeOH. In other words, with less ACN a similar elution efficiency than with MeOH may be achieved. As shown in FIG. 7(c) 50 vol % ACN yields in a good recovery. Follow up experiments have confirmed that from 50 vol % ACN a plateau in elution efficiency is reached. For MeOH, a higher concentration of 80 vol % was necessary to yield a similar elution efficiency.

Beads

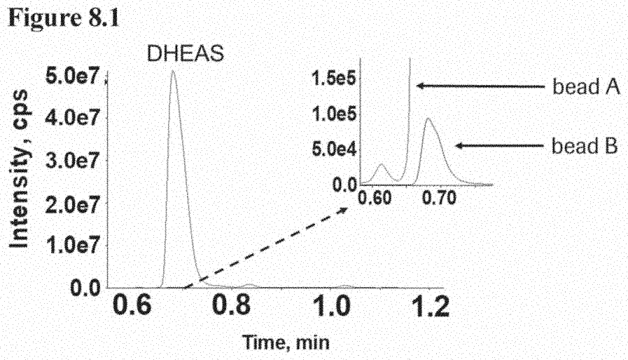

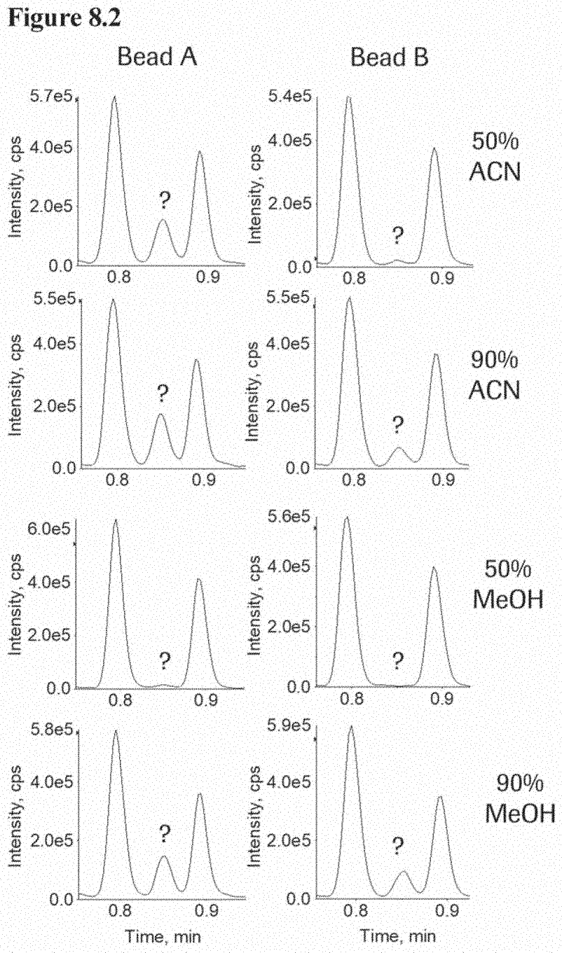

A first evaluation of magnetic beads A and B for the batch-type SPE was already done in Example 5. Example 5 has demonstrated that the magnetic bead type B has a better recovery of 11-oxygenated C19 steroids and most classical androgens detected. The classical androgen DHEAS was better recovered with magnetic beads type A. The DoE performed for the bead elution confirmed this finding. FIG. 8.1 shows an overlay of two chromatograms recorded following sample preparation of bead A and bead B. The DHEAS signal is such high that the bead B chromatogram is barely visible. Moreover, the bead A chromatogram almost reaches detector saturation. Using bead B, the DHEAS signal shrinks down from 5*10⁷ to 1*10⁵ which equals a 500-fold decrease of signal intensity (see magnified spectrum). Pooled serum used for sample preparation contains 2 μg/ml. DHEAS. Facing the fact that endogenous DHEAS levels may deviate up to 6 μg/mL while recovery with magnetic beads type A is better, the risk for a saturation of the beads is increased at such high DHEAS concentrations. Thus, despite a less efficient recovery of DHEAS from a concentration and calibration perspective, magnetic bead type B provides more flexibility. Moreover, due to the high amount of DHEAS found in serum, the loss in recovery by DHEAS is not problematic as it can be corrected by using an ISTD prior to sample preparation.

Influence of Elution Solution and Bead Type on Interferences

Based on the measurements performed in this example it was assessed whether the elution solution and/or the magnetic beads show any differences in interferences. The chromatograms at the bottom of FIG. 8.2 show a MS interference which was detected at the transition from m/z 292.2 to 256.2 of DHEA-¹³C₃ Chromatograms were generated from ACN eluates and from MeOH eluates. Chromatograms are differentiated depending on the relative amount of organic solvent (90% or 50%) and the bead type (A or B). The Figures show three peaks, DHEA-¹³C₃ at Rt=0.8 min. ET-¹³C₃ at Rt=0.9 min and the interfering substance right between them. The interference was generally at higher absolute intensities when bead A was used. Only if 90% organic solvent was applied the interference appears with bead B, however, well below bead A. It was also found that the unknown substance concentration behaves steroid similar, i.e. it increases similar as the concentration of 11OHA4. These results showed that the magnetic beads of type B are advantageous in that they show less interferences. Furthermore, lowering the organic solvent content in the elution buffer to 50 vol % prevents the observed interference significantly.

Summary

Using ACN as elution reagent was found to be advantageous since less organic solvent was required to elute a desired analyte concentration. The data supports that already 50-70 vol % ACN are very efficient in eluting steroid anlaytes.

Regarding the two bead types tested, magnetic bead type B turned out to be more efficient in capturing of 11-oxygenated C19 steroids, T and T-similar substances. However, also bead type B can be used in principle. Only DHEAS recovery is lower with bead type B than with bead type A Due to the high abundance of DHEAS this reduced recovery is even a certain advantage, because saturation is prevented. The loss does not affect method accuracy as it can be compensated by adding an ISTD prior to SPE, which will then be lost to a similar extent. Beads of type B also cause a weaker interfering signal for the detection of DHEA-¹³C₃, which may be used as ISTD.

### Example 7: Concentration of Eluate by Evaporation

Next, it was evaluated to which evaporation after the magnetic bead workflow can further improve sensitivity of the sample preparation method. To this end an enrichment workflow, as established in the preceding examples with evaporation (Evap) was compared to the same workflow without evaporation. The latter is designated as direct injection (DI) herein after.

For both D1 and evaporation, after the first steps of sample preparation (SP), including addition of pretreatment and bead suspension followed by washing steps, beads were treated with a certain amount of elution reagent (EL). In case of the DI workflow, a certain fraction of the supernatant (SN) is transferred to the next vial, diluted with LC-dilute (LC-Dil) and injected to the LCMS system. The dilution was required to reduce organic content so as to permit injection volumes greater than 2-5 μl without peak broadening in LC. Of course, this dilution leads to a further reduction in the concentration of the analytes which is disadvantageous.

For evaporation, after elution the eluate was evaporated to dryness and subsequently resolved in LC-Dil.

Two scenarios (Evap1 and Evap2), which are noted in table 13 below, were tested experimentally. Both were compared with the direct injection (DI) which represents the standard sample prep procedure as used in the preceding examples. Except for the parameters indicated in table 13, the experimental settings were identical. In the first evaporation experiment beads were eluted with 120 μL of 60% ACN while 180 μL were used in the second. Similar amounts minus the bead dead volume (about 20 μL) were transferred, evaporated and re-solubilized into a final volume of 40 μL 20% MeOH which contained internal standards.

TABLE 13
Experimental parameters of evaporation scenarios
(Evap1 and Evap2) and direct injection (DI).

| | V (EL)/ | V (SN)/ | | V (LC-Dil)/ | V (final)/ |
|---|---|---|---|---|---|
| | μL | μL | Evaporation | μL | μL |
| DI | 60 | 30 | No | 30 | 60 |
| Evap1 | 120 | 100 | Yes | 40 | 40 |
| Evap2 | 180 | 160 | Yes | 40 | 40 |

EL: Elution (60% ACN), SN: Supernatant, LC-Dil: LC dilute (20% MeOH + ISTD).

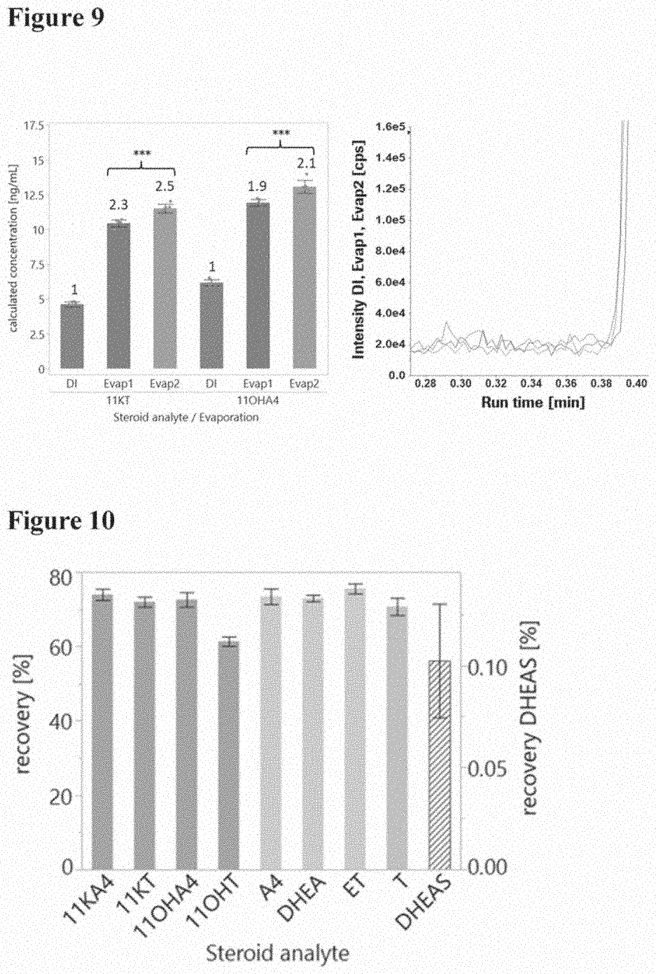

The results of this evaporation experiment are shown in FIG. 9. Exemplary for the set of 11-oxygenated steroids, the concentrations of 11KT and 11OHA4 are graphically displayed on the left. The x-axis differentiates between those two analytes and the type of experiment while the y-axis represents the amount of calculated concentration. Furthermore, bars are labeled by the observed standard enrichment factor (SEF), calculated on the basis of experimental data for each analyte. The SEV was calculated as follows:

$$SEF = \frac{{C}_{Evap}}{{C}_{DI}}$$

The SEF value of DI was defined to be one, since it is the references for SEF calculation. Compared to the reference workflow, evaporation experiments led to a significant increase of the concentration. The second evaporation experiment led to a slightly higher concentration increase. This difference is statistically significant at the α=0.001 level, as indicated by three stars. Evaporation also did not cause baseline increase as evidenced by the TIC chromatograms on the right. This indicates that matrix interferences are not amplified, indicating that by combining the magnetic bead workflow and evaporation increases sensitivity of the method.

The SEF values obtained for other steroids measured (see table 1) were quite similar to those of 11KT and 11OHA4. In average, the SEF gained for 11-oxygenated steroids using Evap1 and Evap2 were 2.1 and 2.3, respectively Evap2 were selected for the experiments shown in the following examples. Evaporation helps to reach lower LOQs. Evaporation did not cause increase of matrix effects which is the major risk of this technique.

### Example 8: Further Method Validation

In the previous examples, individual settings of the sample preparation of the method described herein were varied and improved Table 14 below shows the sample preparation settings selected based on the previous Examples and used in the present example for method validation using spiked samples.

TABLE 14
Summary of sample preparation conditions selected based on
previous examples; Abbreviations: see previous examples.

Final sample prep workflow

| | Content of added | |
|---|---|---|
| Volume/μL | volume | run time/s |
| 150 | Unknown serum sample | 60 |
| 10 | 16X-ISTD mix | |
| 50 | 8% ACN | 50 |
| — | — | — |
| 20 | Bead B | 250 |
| 250 × 2 | 40 mM FA | 65 × 2 |
| 180 | 60% ACN | 275 |
| 160 | 60% ACN | 40 |
| 0 | Evaporation (to dryness) | — |
| 40 | 20% MeOH | — |

The LC settings used were the ones defined in Example 2, above.

Pairing of ISTD and analyte was decided based on retention times in the LC. The ISTD, which eluted closest to the retention time of an analyte was chosen as the quantifying reference standard. Given the retention times in table 15, we chose 11-KT-d₃ as ISTD for the quantification of 11-KT and 11-KA4 and 11OHA4-d₄ as ISTD for 111OHA4 and 11OHT. Furthermore, the ISTD concentration was set to 30% of the highest calibrator (Cal 1). In case of DHEAS the highest calibrator was set to 2000 ng/mL and 200 ng/ml in serum and organic solvent matrix, respectively. All other compounds were set to 20 ng/mL for Cal 1. Consequently, the ISTD levels were set to be 600 ng/ml, and 60 ng/mL in case of DHEAS, depending on the matrix, as well as 6 ng/mL in case of other steroid metabolites for both matrices.

TABLE 15
Elution order of androgenic steroid hormones and corresponding
ISTDs. Retention times were observed on a C18 HPLC column (2.1
mm i.d. × 50 mm) loaded with 2.6 μm fused core particles.

| Analyte | RT [min] | ISTD | RT [min] |
|---|---|---|---|
| 11KA4 | 0.38 | 11KT-d [symbol] | 0.42 |
| 11KT | 0.42 | 11KT-d [symbol] | 0.42 |
| 11OHA4 | 0.48 | 11OHA4-d [symbol] | 0.48 |
| 11OHT | 0.52 | 11OHA4-d [symbol] | 0.48 |
| A4 | 0.68 | A4- [symbol] C [symbol] | 0.68 |
| DHEAS | 0.70 | DHEAS- [symbol] C [symbol] | 0.70 |
| T | 0.75 | T- [symbol] C [symbol] | 0.75 |
| DHEA | 0.84 | DHEA- [symbol] C [symbol] | 0.84 |
| ET | 0.93 | ET- [symbol] C [symbol] | 0.93 |

[symbol]

indicates data missing or illegible when filed

Calibration and LOQ Calculation

LOQs of each analyte were determined using the most abundant transitions in both, neat solution (without the above mentioned sample preparation) and stripped serum matrix using spiked samples. The LOQ calculation was done using JMP software.

The results are depicted in table 16 below

TABLE 16
LOQs, calibration ranges and linearity of androgenic steroids dissolved
in 20% MeOH (without sample preparation) and stripped serum
(including sample preparation). Concentrations are given in ng/mL.

| | 20% MeOH | | | Stripped serum | | |
|---|---|---|---|---|---|---|
| Steroid analyte | LOQ | Cal range | r² | LOQ | Cal range | r² |
| 11OHA4 | 0.1 | 0.01-20 | 0.9997 | 0.1 | 0.01-20 | 0.9996 |
| 11KT | 0.02 | 0.01-20 | 0.9998 | 0.1 | 0.01-20 | 0.9996 |
| 11KA4 | 0.02 | 0.01-20 | 0.9998 | 0.02 | 0.01-20 | 0.9996 |
| 11OHT | 0.1 | 0.01-20 | 0.9992 | 0.1 | 0.01-20 | 0.9991 |
| T | 0.02 | 0.01-20 | 0.9998 | 0.02 | 0.01-20 | 0.9996 |
| A4 | 0.01 | 0.01-20 | 0.9998 | 0.01 | 0.01-20 | 0.9995 |
| DHEA | 1 | 0.01-20 | 0.9995 | 0.2 | 0.01-20 | 0.9983 |
| DHEAS | 0.1 | 0.1-200 | 0.9992 | 100 | 1.0-2000 | 0.9816 |
| ET | 0.01 | 0.01-20 | 0.9998 | 0.02 | 0.01-20 | 0.9995 |

Precision and Accuracy

Precision and accuracy were determined for the most abundant transition of each analyte. The results of these validation experiments are given in the Tables 17 and 18 below. For those analytes measured in the lower calibration range (0.01-20 ng/mL) precision was determined to be below 15% CV. Precision of DHEAS was somewhat higher (17.9% CV) in the highest calibrator but decreased below 10% CV at lower calibrator levels. Acceptable accuracy (<20% error) was calculated from 1-20 ng/mL for the steroids except DHEAS and 100-2000 ng/mL in case of DHEAS. 11-oxygenated steroids 11OHA4, 11KT and 11KA4, were measured very precisely (<10% CV) and very accurately (<5$ error) also from 0.1 ng/ml and over the whole range.

TABLE 17
Method precision determined in spiked stripped serum samples given as CV [%].
Each sample was prepared n = 3 times and measured n = 3 times via LCMS.
DHEAS (*) was measured at 100-fold higher concentration.

| Steroid | Analyte concentration [ng/ml] | | | | | | | |
|---|---|---|---|---|---|---|---|---|
| analyte | 0.01 | 0.02 | 0.1 | 0.2 | 1.0 | 2.0 | 10 | 20 |
| 11OHA4 | <LOQ | <LOQ | 4.07 | 3.78 | 7.00 | 2.24 | 2.70 | 2.87 |
| 11KT | <LOQ | <LOQ | 4.72 | 4.19 | 8.18 | 1.49 | 1.94 | 1.77 |
| 11KA4 | <LOQ | 9.06 | 3.82 | 2.94 | 8.89 | 3.03 | 2.07 | 1.75 |
| 11OHT | <LOQ | <LOQ | 7.29 | 5.93 | 7.58 | 1.98 | 2.55 | 2.22 |
| T | <LOQ | 10.20 | 5.44 | 3.34 | 9.29 | 1.30 | 2.73 | 2.02 |
| A4 | 13.1 | 6.95 | 4.68 | 2.04 | 8.58 | 1.90 | 2.59 | 2.13 |
| DHEA | <LOQ | <LOQ | <LOQ | 10.4 | 10.3 | 4.19 | 4.21 | 3.23 |
| DHEAS* | <LOQ | <LOQ | <LOQ | <LOQ | 9.10 | 9.41 | 7.76 | 17.9 |
| ET | <LOQ | 12.5 | 5.43 | 3.18 | 8.42 | 1.96 | 2.19 | 2.48 |

TABLE 18
Method accuracy determined in spiked stripped serum samples given
as % error [%]. Each sample was prepared n = 5 times and measured
n = 3 times via LCMS. DHEAS (*) was measured at 100-fold higher
concentration. OOS: accuracy is out of spec (20% < % error < −20%)

| Steroid | Analyte concentration [ng/mL] | | | | | | | |
|---|---|---|---|---|---|---|---|---|
| analyte | 0.01 | 0.02 | 0.1 | 0.2 | 1.0 | 2.0 | 10 | 20 |
| 11OHA4 | <LOQ | <LOQ | −0.56 | −1.80 | −0.80 | 0.63 | −1.10 | −0.10 |
| 11KT | <LOQ | <LOQ | −3.10 | −0.90 | −0.30 | 0.24 | 0.03 | −0.10 |
| 11KA4 | <LOQ | 1.58 | −0.50 | −0.20 | 0.02 | 0.05 | −0.10 | 0.00 |
| 11OHT | <LOQ | <LOQ | OOS | −17.00 | −1.20 | 2.19 | 0.84 | −0.50 |
| T | <LOQ | OOS | 17.47 | 8.20 | 3.79 | 3.87 | 3.60 | 1.63 |
| A4 | OOS | OOS | 10.25 | 4.23 | 1.49 | 3.60 | 4.47 | 2.40 |
| DHEA | <LOQ | <LOQ | <LOQ | 5.73 | 4.47 | 5.50 | 4.32 | 3.23 |
| DHEAS* | <LOQ | <LOQ | <LOQ | <LOQ | −3.88 | 5.68 | 13.55 | 14.01 |
| ET | OOS | OOS | −5.80 | −4.50 | −1.00 | 0.50 | 0.54 | −0.10 |

Recovery and Enrichment Factor of Steroids

The recovery of androgenic steroids by the sample preparation as indicated above was assessed. The results are plotted in the bar chart of FIG. 10. The enrichment factors compared to the sample started from (i.e. the increase in the concentration of the analyte in the solution obtained from the sample preparation vs the initial sample subjected to sample preparation started with) are given in table 19, below. The right y-axis in the bar chart of FIG. 10 indicates the recovery of DHEAS only. The left y-axis applies to all other analytes. The bar heights indicate that most analytes were recovered by 70% to 75%. 11OHT exhibits recovery values which are ca 10% lower than other 11-oxygenated steroid hormones. In case of DHEAS, only 0.1% were recovered. For those steroids with high recovery a threefold enhancement of the initial serum concentration was achieved.

All analytes other than DHEAS were recovered by 60% to 80%. The much lower recovery of DHEAS is not problematic since DHEAS occurs in serum samples typically at much higher concentrations. The lower recovery can even have the advantage that the system is not saturated or contaminated with extremely high DHEAS amounts. Quantification is not affected since the ISTD also shows a similar recovery. The threefold enrichment of serum androgens is extremely helpful to meet endogenous androgen levels. The lower recovery for 11OHT implies that 11OHA4-d₄ does not ideally fulfill the ISTD prerequisites for the quantification of 11OHT. Using deuterium—or ^{—}C-labeled 11OHT standards may improve this result.

TABLE 19
Increase in the concentration of the analyte in the solution
obtained from the sample preparation vs. the initial sample
subjected to sample preparation (enrichment factor).

Enrichment factor

| Analyte | Mean | CV [%] |
|---|---|---|
| 11KA4 | 3.0 | 2.1 |
| 11KT | 2.9 | 1.9 |
| 11OHA4 | 2.9 | 2.7 |
| 11OHT | 2.5 | 2.1 |
| A4 | 2.9 | 2.9 |
| DHEA | 2.9 | 1.2 |
| ET | 3.0 | 1.8 |
| T | 2.8 | 3.3 |
| DHEAS | 0.004 | 27.2 |

### Example 9: Application of the Method to Samples From Healthy Individuals and PCOS Patients

Quantification of androgen metabolites including 11-oxygenated steroids in serum of PCOS patients (n=20) and control subjects (n=22) following the workflow developed in the preceding examples was performed. Sample preparation was conducted as shown in table 14, above. Data processing and peak integration was conducted as described above. Some donor samples showed that there can be interferences hampering the detection of some analytes with a defined peak. In these cases the other of the two selected peak was used for quantification. Thus, it was still possible to identify and reliable quantify the analytes. Obvious outliers occurring the measurement of some individual patients were excluded.

The results (see table 20 below) demonstrate that detection of 11-oxygenated steroids as well as the other steroids tested was possible with the method as developed herein both in healthy controls and PCOS patients.

TABLE 20
Observed serum levels of androgenic steroids in n = 22
controls and n = 20 patients suffering from PCOS. P-
values were calculated following Wilcoxon two-sample test,
excluding outliers. Data are given as median and IQR (in brackets).

| [ng/mL] | Controls | | PCOS | | p-value |
|---|---|---|---|---|---|
| 11OHA4 | 0.9 | (0.6-1.2) | 1.2 | (0.8-1.7) | 0.1 |
| 11KT | 0.2 | (0.2-0.3) | 0.3 | (0.3-0.4) | 0.003 |
| 11KA4 | 0.1 | (0.09-0.2) | 0.1 | (0.08-0.2) | 0.9 |
| 11OHT | 0.1 | (0.1-0.2) | 0.1 | (0.1-0.2) | 0.2 |
| T | 0.2 | (0.2-0.3) | 0.5 | (0.3-0.6) | <0.0001 |
| A4 | 0.7 | (0.5-1.0) | 1.8 | (1.4-2.6) | <0.0001 |
| DHEA | 2.6 | (1.8-3.3) | 4.7 | (3.1-5.7) | 0.0005 |
| DHEAS | 1362 | (1012-1891) | 2436 | (1734-3126) | 0.002 |

This patent application claims the priority of the European patent application 20215188.2, wherein the content of this European patent application is hereby incorporated by references.

1. A method for detecting or quantifying one or more steroids in a sample using mass spectrometry, said method comprising:
a) extracting the one or more steroids from the sample using solid phase extraction (SPE) so as to obtain an SPE extract comprising the one or more steroids;
b) concentrating the one or more steroids, said concentrating comprising evaporating solvent from the SPE-extract obtained in a); and
c) detecting or quantifying the one or more steroids in the sample using mass spectrometry,
wherein the one or more steroids comprise one or more 11-oxygenated C19 steroids.

2. The method of claim 1, wherein the one or more 11-oxygenated C19 steroids are selected from the group consisting of 11β-Hydroxyandrostenedione (11-OHA4), 11-Ketotestosterone (11KT), 11-Ketoandrostenedione (11KA4), and 11β-Hydroxytestosterone (11OHT).

3. The method of claim 1, wherein the one or more steroids comprise 11β-Hydroxyandrostenedione (11-OHA4), 11-Ketotestosterone (11KT), 11-Ketoandrostenedione (11KA4), 11β-Hydroxytestosterone (11OHT), Testosterone (T), Androstenedione (A4), Dehydroepiandrosterone (DHEA), Dehydroepiandrosterone sulfate (DHEAS), and Epitestosterone (ET).

4. The method of claim 1, wherein the sample is serum or plasma.

5. The method of claim 1, wherein the sample volume subjected to the SPE in a) is less than 500 μl.

6. The method of claim 1, wherein the SPE comprises:
d) capturing the one or more steroids to a solid phase;
e) optionally one or more wash steps; and
f) eluting the one or more steroids from the solid phase.

7. The method of claim 6, wherein the SPE comprises the one or more wash steps, and wherein said one or more wash steps use a wash solution having a pH of 2 to 4.

8. The method of claim 1, wherein magnetic particles are the solid phase used for the SPE.

9. The method of claim 1, wherein the solid phase of the SPE is coated with a polymer matrix comprising at least one positively charged nitrogen atom.

10. The method of claim 1, wherein said evaporating of the solvent of the SPE extract results in a liquid volume reduction of 50 to 100%.

11. The method of claim 1, wherein the mass spectrometry is a LC-MS analysis.

12. The method of claim 11, wherein the liquid chromatography (LC) is HPLC.

13. The method of claim 11, wherein the LC is a RP-HPLC and the RP-HPLC comprises a gradient elution.

14. The method of claim 1, wherein
(i) the one or more steroids comprise 11OHA4, and wherein for 11OHA4 a positively charged parent ion having an m/z value of 303.1±0.5 is generated and selected for fragmentation, and wherein a first positively charged 11OHA4-fragment ion having an m/z ratio of 267.2±0.5 and/or a second positively charged 11OHA4-fragment ion having an m/z ratio of 121.1±0.5 are generated by fragmentation of the parent ion selected for 11OHA4 and wherein said first and/or second 11OHA4-fragment ion(s) is/are detected by the mass spectrometry and is/are optionally used for quantification;
(ii) the one or more steroids comprise 11KT, and wherein for 11KT a positively charged parent ion having an m/z value of 303.1±0.5 is generated and selected for fragmentation, and wherein a first positively charged 11KT-fragment ion having an m/z ratio of 121.1±0.5 and/or a second positively charged 11KT-fragment ion having an m/z ratio of 259.2±0.5 are generated by fragmentation of the parent ion selected for 11KT, and wherein said first and/or second 11KT-fragment ion is/are detected by the mass spectrometry and is/are optionally used for quantification;
(iii) the one or more steroids comprise 1KA, and wherein for 11KA a positively charged parent ion having an m/z value of 301.05±0.50 is generated and selected for fragmentation, and wherein a first positively charged 11KA-fragment ion having an m/z ratio of 257.2±0.5 and/or a second positively charged 11KA-fragment ion having an m/z ratio of 121.2±0.5 are generated by fragmentation of the parent ion selected for 11KA, and wherein said first and/or second 11KA-fragment ion is/are detected in the by the mass spectrometry and is/are optionally used for quantification;
(iv) the one or more steroids comprise 11OHT, and wherein for 11OHT a positively charged parent ion having an m/z value of 305.2±0.5 is generated and selected for fragmentation, and wherein a first positively charged 11OHT-fragment ion having an m/z ratio of 269.2±0.5 and/or a second positively charged 11OHT-fragment ion having an m/z ratio of 121.1±0.5 are generated by fragmentation of the parent ion selected for 11OHT, and wherein said first and/or second 11OHT-fragment ion is/are detected in the by the mass spectrometry and is/are optionally used for quantification;
(v) the one or more steroids comprise T, and wherein for T a positively charged parent ion having an m/z value of 289.2±0.5 is generated and selected for fragmentation, and wherein a first positively charged T-fragment ion having an m/z ratio of 97.1±0.5 and/or a second positively charged T-fragment ion having an m/z ratio of 109.1±0.5 are generated by fragmentation of the parent ion selected for T, and wherein said first and/or second T-fragment ion is/are detected by the mass spectrometry and is/are optionally used for quantification;
(vi) the one or more steroids comprise A4, and wherein for A4 a positively charged parent ion having an m/z value of 287.2±0.5 is generated and selected for fragmentation, and wherein a first positively charged A4-fragment ion having an m/z ratio of 97.1±0.5 and/or a second positively charged A4-fragment ion having an m/z ratio of 109.1±0.5 are generated by fragmentation of the parent ion selected for A4, and wherein said first and/or second A4-fragment ion is/are detected by the mass spectrometry and is/are optionally used for quantification;
(vii) the one or more steroids comprise DHEA, and wherein for DHEA a positively charged parent ion having an m/z value of 289.2±0.5 is generated and selected for fragmentation, and wherein a first positively charged DHEA-fragment ion having an m/z ratio of 213.1±0.5 and/or a second positively charged DHEA-fragment ion having an m/z ratio of 91.0±0.5 are generated by fragmentation of the parent ion selected for DHEA, and wherein said first and/or second DHEA-fragment ion is/are detected by the mass spectrometry and is/are optionally used for quantification;
(viii) the one or more steroids comprise DHEAS, and wherein for DHEAS a negatively charged parent ion having an m/z value of 367.2±0.5 is generated and selected for fragmentation, and wherein a first negatively charged DHEAS-fragment ion having an m/z ratio of 80.1±0.5 and/or a second negatively charged DHEAS-fragment ion having an m/z ratio of 97.1±0.5 are generated by fragmentation of the parent ion selected for DHEAS, and wherein said first and/or second DHEAS-fragment ion is/are detected by the mass spectrometry and is/are optionally used for quantification; and/or
(ix) the one or more steroids comprise ET, and wherein for ET a positively charged parent ion having an m/z value of 289.2±0.5 is generated and selected for fragmentation, and wherein a first positively charged ET-fragment ion having an m/z ratio of 109.0±0.5 and/or a second positively charged ET-fragment ion having an m/z ratio of 97.0±0.5 are generated by fragmentation of the parent ion selected for ET, and wherein said first and/or second ET-fragment ion is/are detected by the mass spectrometry and is/are optionally used for quantification.

15. The method of claim 1, wherein the method is automated.

16. The method of claim 5, wherein the sample volume subjected to the SPE in a) is less than 250 μl or less than 200 μl.

17. The method of claim 10, wherein said evaporating of the solvent of the SPE extract results in a liquid volume reduction of 60 to 85%.

18. The method of claim 11, wherein the mass spectrometry is a LC-MS/MS analysis.

19. The method of claim 12, wherein the liquid chromatography (LC) is reversed phase HPLC (RP-HPLC).

20. The method of claim 19, wherein the liquid chromatography (LC) is a C18-HPLC.
